(19) United States
(12) Patent Application Publication
(10) Pub. No.: US 2017/0087221 A1
(43) Pub. Date: Mar. 30, 2017

(54) NITRIC OXIDE-MODULATING BONE-TARGETING COMPLEXES

(71) Applicant: Elwha LLC, Bellevue, WA (US)

(72) Inventors: Mahalaxmi Gita Bangera, Renton, WA (US); Roderick A. Hyde, Redmond, WA (US); Muriel Y. Ishikawa, Livermore, CA (US); Eric C. Leuthardt, St. Louis, MO (US); Lowell L. Wood, JR., Bellevue, WA (US)

(21) Appl. No.: 14/870,465

(22) Filed: Sep. 30, 2015

(51) Int. Cl. A61K 38/44; C12N 9/02; C12N 9/96; A61K 47/48

(52) U.S. Cl. CPC A61K 38/44; A61K 47/48338; A61K 47/48015; A61K 47/48246; C12N 9/0075; C12N 9/96; C12Y 114/13039

(57) ABSTRACT

Compositions and methods are described herein for treating a bone disorder, including a bone-targeting complex including at least a portion of a nitric oxide synthase; a bone-targeting agent; and a linker coupling the at least a portion of the nitric oxide synthase to the bone-targeting agent.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a composition includes, but is not limited to, a bone-targeting complex including an inhibitor of nitric oxide synthase uncoupling; a bone-targeting agent; and a linker coupling the inhibitor of nitric oxide synthase uncoupling to the bone-targeting agent. In addition to the foregoing, other composition aspects are described in the claims, drawings, and text forming a part of the present disclosure.

In an aspect, a composition includes, but is not limited to, a bone-targeting complex including an inhibitor of nitric oxide synthase uncoupling; and a bone-targeting agent associated with the inhibitor of nitric oxide synthase uncoupling. In addition to the foregoing, other composition aspects are described in the claims, drawings, and text forming a part of the present disclosure.

In an aspect, a method of treating a bone disorder includes, but is not limited to, administering a bone-targeting complex to a subject in need of treatment for a bone disorder, the bone-targeting complex including an inhibitor of nitric oxide synthase uncoupling, a bone-targeting agent, and a linker coupling the inhibitor of nitric oxide synthase uncoupling to the bone-targeting agent. In addition to the foregoing, other method aspects are described in the claims, drawings, and text forming a part of the present disclosure.

In an aspect, a composition includes, but is not limited to, a bone-targeting complex including an activator of nitric oxide synthase; a bone-targeting agent; and a linker coupling the activator of nitric oxide synthase to the bone-targeting agent. In an embodiment, the linker comprises a cleavable linker. In addition to the foregoing, other composition aspects are described in the claims, drawings, and text forming a part of the present disclosure.

In an aspect, a method of treating a bone disorder includes, but is not limited to, administering a bone-targeting complex to a subject in need of treatment for a bone disorder, the bone-targeting complex including an activator of nitric oxide synthase, a bone-targeting agent, and a linker coupling the activator of nitric oxide synthase to the bone-targeting agent. In addition to the foregoing, other method aspects are described in the claims, drawings, and text forming a part of the present disclosure.

In an aspect, a composition includes, but is not limited to, a bone-targeting complex including an activator of nitric oxide synthase, and a bone-targeting agent. In addition to the foregoing, other composition aspects are described in the claims, drawings, and text forming a part of the present disclosure.

In an aspect, a composition includes, but is not limited to, a bone-targeting complex including at least a portion of a nitric oxide synthase, a bone-targeting agent, and a linker coupling the at least a portion of the nitric oxide synthase to the bone-targeting agent. In addition to the foregoing, other composition aspects are described in the claims, drawings, and text forming a part of the present disclosure.

In an aspect, a composition includes, but is not limited to, a bone-targeting complex including at least a portion of a nitric oxide synthase, a bone-targeting agent, and a cell-penetrating means. In addition to the foregoing, other composition aspects are described in the claims, drawings, and text forming a part of the present disclosure.

In an aspect, a method of treating a bone disorder includes, but is not limited to, administering a bone-targeting complex to a subject in need of treatment for a bone disorder, the bone-targeting complex including at least a portion of a nitric oxide synthase, a bone-targeting agent, and a linker coupling the at least a portion of the nitric oxide synthase to the bone-targeting agent. In addition to the foregoing, other method aspects are described in the claims, drawings, and text forming a part of the present disclosure.

In an aspect, a method of treating a bone disorder includes, but is not limited to, administering a bone-targeting complex to a subject in need of treatment for a bone disorder, the bone-targeting complex including at least a portion of a nitric oxide synthase, a bone-targeting agent, and a cell-penetrating means. In addition to the foregoing, other method aspects are described in the claims, drawings, and text forming a part of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a block diagram of a bone-targeting complex including an inhibitor of nitric oxide synthase uncoupling, a bone-targeting agent, and a linker.

FIG. 2 is a block diagram illustrating further aspects of a bone-targeting complex such as shown in FIG. 1.

FIG. 3 is a block diagram showing further aspects of a bone-targeting complex such as depicted in FIG. 1.

FIG. 4 is a block diagram depicting further aspects of a bone-targeting complex such as illustrated in FIG. 1.

FIG. 5 is a block diagram of a method of treating a bone disorder.

FIG. 6 is a block diagram of a bone-targeting complex including an inhibitor of nitric oxide synthase uncoupling and a bone-targeting agent.

FIG. 7 is a block diagram of a bone-targeting complex including an activator of nitric oxide synthase, a bone-targeting agent, and a linker.

FIG. 8 is a block diagram illustrating further aspects of a bone-targeting complex such as shown in FIG. 7.

FIG. 9 is a block diagram showing further aspects of a bone-targeting complex such as depicted in FIG. 7.

FIG. 10 is a block diagram depicting further aspects of a bone-targeting complex such as illustrated in FIG. 7.

FIG. 11 is a block diagram illustrating further aspects of a bone-targeting complex such as shown in FIG. 7.

FIG. 12 is a block diagram of a method of treating a bone disorder.

FIG. 13 is a block diagram of a bone-targeting complex including an activator of nitric oxide synthase and a bone-targeting agent.

FIG. 14 is a block diagram of a bone-targeting complex including at least a portion of nitric oxide synthase, a bone-targeting agent, and a linker.

FIG. 15 is a block diagram illustrating further aspects of a bone-targeting complex such as shown in FIG. 14.

FIG. 16 is a block diagram showing further aspects of a bone-targeting complex such as depicted in FIG. 14.

FIG. 17 is a block diagram depicting further aspects of a bone-targeting complex such as illustrated in FIG. 14.

FIG. 18 is a block diagram illustrating further aspects of a bone-targeting complex such as shown in FIG. 14.

FIG. 19 is a block diagram showing further aspects of a bone-targeting complex such as depicted in FIG. 14.

FIG. 20 is a block diagram of a bone-targeting complex including at least a portion of nitric oxide synthase, a bone-targeting agent, a linker, and a cell-penetrating means.

FIG. 21 shows a block diagram of a method of treating a bone disorder.

FIG. 22 shows a block diagram of a method of treating a bone disorder.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Described herein are compositions and methods for treating a bone disorder with a bone-targeting complex that includes a first agent with bone-targeting properties to direct the bone-targeting complex to bone tissue or cells and a second agent with properties to promote or prolong the activity of nitric oxide synthase. Nitric oxide synthase (NOS; L-arginine, NADPH:oxygen oxidoreductases, NO forming; EC 1.14.13.39) is an enzyme that generates the second messenger nitric oxide (NO). There are three isozymes of nitric oxide synthase referred to as neuronal nNOS (or NOS I), inducible iNOS (or NOS II), and endothelial eNOS (or NOS III). All isozymes of nitric oxide synthase use L-arginine as a substrate, as well as co-substrates molecular oxygen and reduced nicotinamide-adenine-dinucleotide phosphate (NADPH). Flavin adenine dinucleotide (FAD), flavin mononucleotide (FMN), and (6R-)5,6,7,8-tetrahydro-L-biopterin (BH4) are cofactors. Nitric oxide synthase is fully functionally to generate NO when in a homodimer configuration. See, e.g., Andrew & Mayer (1999) “Enzymatic function of nitric oxide synthases,” Cardiovascular Research, 43:521-531; and Forestermann & Sessa (2012) “Nitric oxide synthases: regulation and function,” Eur. Heart J., 33:829-837, which are incorporated herein by reference. Under certain conditions (e.g., in the absence of arginine and/or BH4) nitric oxide synthase losses the ability to convert L-arginine to L-citrulline to generate NO and instead removes an electron from NADPH and donates it to molecular oxygen to yield superoxide. This “uncoupled” nitric oxide synthase leads to a state of oxidative stress. Increased oxidation of BH4 may be one mechanism by which uncoupling of nitric oxide synthase is triggered. Preventing or reversing the uncoupling of nitric oxide synthase can be used to increase NO production. See, e.g., Roe & Ren (2012) “Nitric oxide synthase uncoupling: A therapeutic target in cardiovascular diseases,” Vasc. Pharm. 57:168-172, which is incorporated herein by reference.

NO has been implicated in numerous biological pathways including those associated with bone. More specifically, NO has been shown to suppress osteoclast bone resorption and promote growth of osteoblasts. See, e.g., Wimalawansa (2010) “Nitric oxide and bone,” Ann. N. Y. Acad. Sci., 1192:394-406; Hamilton et al. (2013) “Organic nitrates for osteoporosis: an update,” BoneKEy Reports 2, Article No: 259; Van't Hof & Ralston (2001) “Nitric oxide and bone,” Immunol. 103:255-261, which are incorporated herein by reference. As such, increasing the production of NO by promoting nitric oxide synthase activity is a potential means of treating bone related disorders, e.g., osteoporosis. Described herein are compositions and methods for treating bone related disorders by promoting nitric oxide synthase activity.

Described herein are embodiments of a composition including a bone-targeting complex. In some embodiments, the bone-targeting complex includes an inhibitor of nitric oxide synthase uncoupling, a bone-targeting agent, and a linker coupling the inhibitor of nitric oxide synthase uncoupling to the bone-targeting agent.

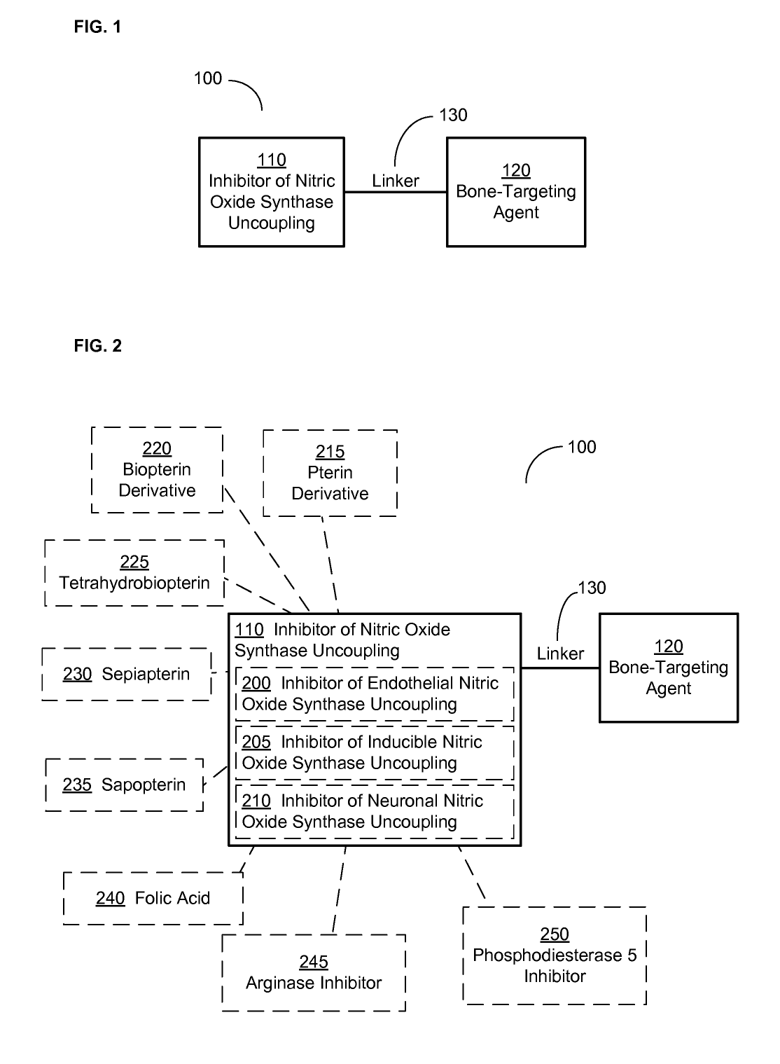

FIG. 1 illustrates aspects of a composition including a bone-targeting complex with an inhibitor of nitric oxide synthase uncoupling and a bone-targeting agent. FIG. 1 shows a block diagram of bone-targeting complex 100 including an inhibitor of nitric oxide synthase uncoupling 110, a bone-targeting agent 120, and a linker 130 coupling the inhibitor of nitric oxide synthase uncoupling 110 to the bone-targeting agent 120.

Bone-targeting complex 100 includes an inhibitor of nitric oxide synthase uncoupling 110. In some embodiments, the inhibitor of nitric oxide synthase uncoupling is configured to or has the properties of preventing the uncoupling of nitric oxide synthase and as such prolonging the nitric oxide (NO) generating activity of the nitric oxide synthase. In some embodiments, the inhibitor of nitric oxide synthase uncoupling is configured to or has the properties of restoring nitric oxide synthase to a coupled form and as such restoring the nitric oxide (NO) generating activity of the nitric oxide synthase. Preventing the uncoupling of nitric oxide synthase or restoring the coupling of nitric oxide synthase also prevents the generation of damaging superoxides by uncoupled nitric oxide synthase.

FIG. 2 illustrates further aspects of bone-targeting complex 100. FIG. 2 shows a block diagram of bone-targeting complex 100 including inhibitor of nitric oxide synthase uncoupling 110, bone-targeting agent 120, and linker 130. In an aspect, the inhibitor of nitric oxide synthase uncoupling is configured to or has the properties of preventing the uncoupling of at least one of endothelial, inducible, or neuronal nitric oxide synthase. In an aspect, the inhibitor of nitric oxide synthase uncoupling is configured to or has the properties of restoring at least one of endothelial, inducible, or neuronal nitric oxide synthase to a coupled form. In an aspect, the inhibitor of nitric oxide synthase uncoupling comprises an inhibitor of endothelial nitric oxide uncoupling 200. In an aspect, the inhibitor of nitric oxide synthase uncoupling comprises an inhibitor of inducible nitric oxide synthase uncoupling 205. In an aspect, the inhibitor of nitric oxide synthase uncoupling comprises an inhibitor of neuronal nitric oxide synthase uncoupling 210.

Also shown in FIG. 2 are non-limiting embodiments of inhibitors of nitric oxide synthase uncoupling. In an aspect, the inhibitor of nitric oxide synthase uncoupling 110 comprises a pterin derivative 215. For example, a bone-targeting complex can include a pterin [2-aminopteridin-4(3H)-one] derivative associated with a bone-targeting agent, e.g., bisphosphonate, through a linker. In an aspect, the inhibitor of nitric oxide synthase uncoupling includes ciliapterin, neopterin, limipterin, tepidopterin, 6-hydroxymethylpterin, 6-(pentahydroxypentyl)-pterin, 6-hydroxymethyl-8-methylisoxanthopterin, or asperopterin-A. See, e.g., Hanaya & Yamamoto (2013) “Synthesis of Biopterin and Related Pterin Glycosides,” IUBMB Life 65:300-309, which is incorporated herein by reference.

In an aspect, the inhibitor of nitric oxide synthase uncoupling 110 comprises a biopterin derivative 220. For example, the biopterin derivative can include biopterin, D-biopterin, orinapterin, L-threoneopterin, neopterin, umanopterin, primapterin, 2-amino-4-hydroxy-6-pteridinecarboxylic acid, pterin, or isoxanthopterin. In an aspect, the inhibitor of nitric oxide synthase uncoupling includes D-biopterin (2-amino-6-[(1R,2S)-1,2-dihydroxypropyl]-1,4-dihydropteridin-4-one). In an aspect, the biopterin derivative includes an analog of biopterin. In an aspect, the biopterin derivative includes a pterin analog. See, e.g., U.S. Pat. No. 8,324,210 to Kakkis titled “Pterin Analogs,” which is incorporated herein by reference.

In an aspect, the inhibitor of nitric oxide synthase uncoupling 110 comprises tetrahydrobiopterin (BH4) 225. For example, the bone-targeting complex can include BH4 (2-amino-6-(1,2-dihydroxypropyl)-1,4,5,6,7,8-hexahydropteridin-4-one) associated with a bone-targeting agent, e.g., bisphosphonate, through a linker. In an aspect, the inhibitor of nitric oxide synthase uncoupling includes a precursor and/or derivative of tetrahydrobiopterin. For example, the inhibitor of nitric oxide synthase uncoupling can include O2′-4a-cyclic-tetrahydrobiopterin, 4a-carbinolamine tetrahydrobiopterin, sapropterin, L-erythro-tetrahydrobiopterin, 4a-hydroxytetrahydrobiopterin, 6-methyltetrahydropterin, or similar compounds.

In an aspect, the inhibitor of nitric oxide synthase uncoupling 110 comprises sepiapterin 230. For example, the bone-targeting complex can include sepiapterin (2-amino-6-[(2S)-2-hydroxypropanoyl]-7,8-dihydro-1H-pteridin-4-one), a stable precursor of tetrahydrobiopterin, associated with a bone-targeting agent, e.g., bisphosphonate, through a linker. See, e.g., Jo et al. (2011) “Inhibition of nitric oxide synthase uncoupling by sepiapterin improves left ventricular function in streptozotocin-induced diabetic mice,” Clin. Exp. Pharm. Physiol. 38:485-493, which is incorporated herein by reference.

In an aspect, the inhibitor of nitric oxide synthase uncoupling 110 comprises sapopterin 235. For example, the bone-targeting complex can include sapopterin ((6R)-2-amino-6-[(1R,2S)-1,2-dihydroxypropyl]-5,6,7,8-tetrahydro-4(1H)-pteridinone), a synthetic preparation of tetrahydrobiopterin, associated with a bone-targeting agent, e.g., bisphosphonate, through a linker.

In an aspect, the inhibitor of nitric oxide synthase uncoupling 110 comprises folic acid 240. For example, the bone-targeting complex can include folic acid associated with a bone-targeting agent, e.g., a hydroxyapatite-binding polypeptide. See, e.g., Stroes et al. (2000) “Folic acid reverts dysfunction of endothelial nitric oxide synthase,” Circulation Res. 86:1129-1134; and Roe et al. (2013) “Folic acid reverses nitric oxide synthase uncoupling and prevents cardiac dysfunction in insulin resistance: Role of Ca(2+)/calmodulin-activated protein kinase II,” Free Radical Biol. Med., 65:234-243, which are incorporated herein by reference.

In an aspect, the inhibitor of nitric oxide synthase uncoupling 110 comprises an arginase inhibitor 245. For example, inhibition of arginase increases the availability of arginine as a substrate for nitric oxide synthase and inhibits, prevents, and/or reverses uncoupling of nitric oxide synthase activity. In an aspect, the arginase inhibitor includes ornithine. See, e.g., U.S. Pat. No. 5,767,160 to Kaesemeyer titled “Method and Formulation of Stimulating Nitric Oxide Synthesis,” which is incorporated herein by reference. In an aspect, the arginase inhibitor includes N-hydroxy-guanidinium derivatives; boronic acid derivatives (e.g., 2(S)-amino-6-boronohexanoic acid and S-(2-boronoethyl)-1-cysteine (BEC)), and (R)-2-amino-6-borono-2-(2-(piperidin-1-yl)ethyl)hexanoic acid. See, e.g., Steppan et al. (2013) “Development of novel arginase inhibitors for therapy of endothelial dysfunction,” Front. Immunol., September 17; 4:278. doi: 10.3389/fimmu.2013.00278, which is incorporated herein by reference. Additional non-limiting examples of inhibitors of arginase activity are described in U.S. Pat. No. 6,387,890 to Christianson et al. titled “Compositions and Methods for Inhibiting Arginase Activity;” and in U.S. Pat. No. 6,723,710 to Christianson et al. titled “Compositions for Inhibiting Arginase Activity,” which are incorporated herein by reference.

In an aspect, the inhibitor of nitric oxide synthase uncoupling 110 comprises a phosphodiesterase 5 inhibitor 250. In an aspect, the inhibitor of nitric oxide synthase uncoupling includes a cyclic GMP-specific phosphodiesterase 5 inhibitor. In an aspect, the inhibitor of nitric oxide synthase uncoupling includes a PDE5 inhibitor. In an aspect, a PDE5 inhibitor reverses uncoupling of nitric oxide synthase. See, e.g., Bivalacqua et al. (2013) “Sildenafil Citrate-Restored eNOS and PDE5 Regulation in Sickle Cell Mouse Penis Prevents Priapism Via Control of Oxidative/Nitrosative Stress,” PLoS ONE 8(7) e:68028, which is incorporated herein by reference. In an aspect, the phosphodiesterase 5 inhibitor includes sildenafil, tadalafil, vardenafil, udenafil, avanafil, mirodenafil, dasantafil, or other inhibitor of PDE5. In an aspect, the inhibitor of nitric oxide synthase uncoupling includes at least one of a PDE1 inhibitor, a PDE2 inhibitor, a PDE3 inhibitor, a PDE4 inhibitor, a PDE6 inhibitor, a PDE7 inhibitor, a PDE8 inhibitor, a PDE9 inhibitor, a PDE10 inhibitor, and/or a PDE11 inhibitor. Non-limiting examples of PDE inhibitors include caffeine, aminophylline, 3-isobutyl-1-methylxanthine (IBMX), paraxanthine, pentoxyfylline, theobromine, theophylline, vinpocetine, erythro-9-(2-hydroxy-3-nonyl)adenine (EHNA), BAY 60-7550, oxindole, inamrinone, milrinone, enoximone, cilostazol, rolipram, ibudilast, piclamilast, drotaverine, rofumilast, apremilast, tofimilast, dipyridamole, papaverine, zaprinast, zardaverine, vesnarinone, and the like.

Bone-targeting complex 100 includes a bone-targeting agent 120. The bone-targeting agent is configured to or has the properties of selectively accumulating in bone tissue and cells. In an aspect, the bone-targeting agent includes an osteotropic agent. In an aspect, the bone-targeting agent includes a “targetor” moiety able to recognize bones cells or components thereof.

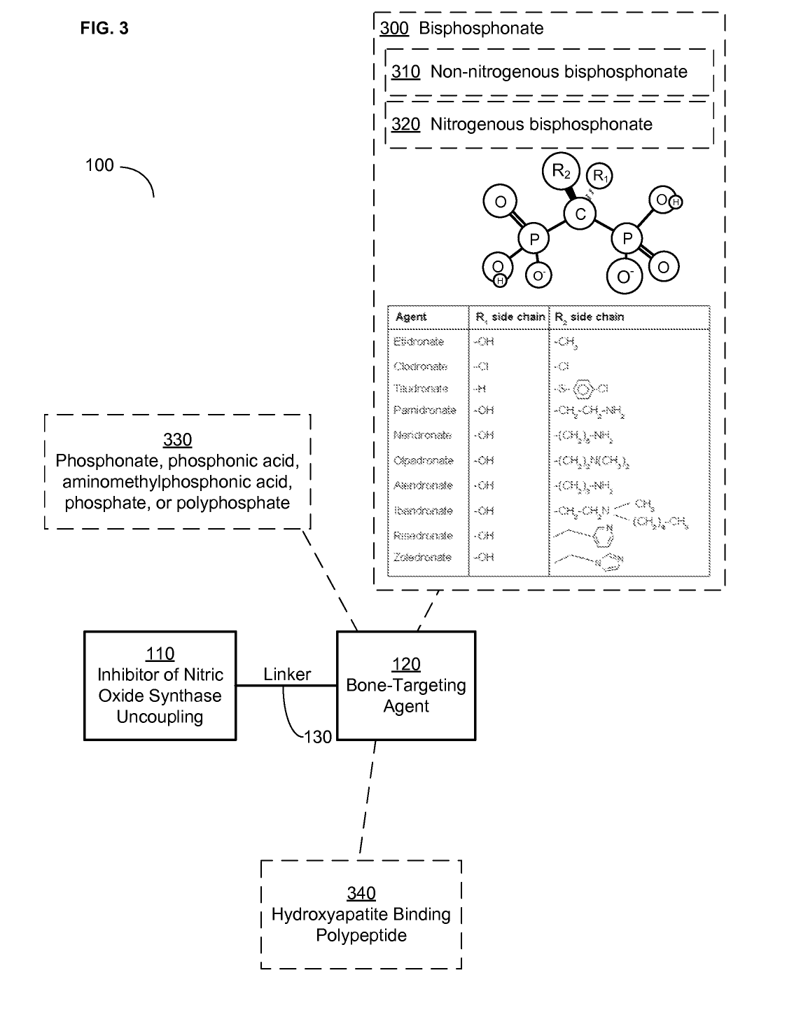

FIG. 3 illustrates further aspects of bone-targeting complex 100. FIG. 3 shows a block diagram of bone-targeting complex 100 including inhibitor of nitric oxide synthase uncoupling 110, bone-targeting agent 120, and linker 130. Also shown are non-limiting embodiments of bone-targeting agents.

In an aspect, the bone-targeting agent 120 comprises bisphosphonate 300. Bisphosphonates are chemically stable derivatives of inorganic pyrophosphate (PPi), have a very high affinity for bone mineral, e.g., hydroxyapatite, and are preferentially incorporated into sites of active bone remodeling. See, e.g., Drake et al. (2008) “Bisphosphonates: Mechanism of action and role in clinical practice,” Mayo Clin. Proc. 83:1032-1045, which is incorporated herein by reference. In an aspect, the bone-targeting agent comprises a non-nitrogenous bisphosphonate 310. Non-limiting examples of non-nitrogenous or non-nitrogen-containing bisphosphonates include etidronate, clodronate, and tiludronate. In an aspect, the bone-targeting agent comprises a nitrogenous bisphosphonate 320. Non-limiting examples of nitrogenous bisphosphonates include pamidronate, neridronate, olpadronate, alendronate, ibandronate, risedronate, and zoledronate. In an aspect, a nitrogenous bisphosphonate can be conjugated through as associated amino group to another moiety, e.g., an inhibitor of nitric oxide synthase uncoupling. For example, Pignatello et al. describe conjugation of a moiety, e.g., poly(lactide-co-glycolide) (PLGA) to an amino group of the bisphosphonate alendronate. See, Pignatello et al. (2012) “Synthesis and Biological Evaluation of a New Polymeric Conjugate and Nanocarrier with Osteotropic Properties,” J. Funct. Biomater. 3:79-99, which is incorporated herein by reference.

In an aspect, the bone-targeting agent 120 comprises an organic phosphate. In an aspect, the bone-targeting agent 120 comprises phosphonate, phosphonic acid, aminomethylphosphonic acid, phosphate, or polyphosphate, as shown in block 330. In an aspect, the bone-targeting agent includes sodium orthophosphate or hydroxyethylidene diphosphonate. In an aspect, the bone-targeting agent includes a phosphate derivative. For example, the bone-targeting agent can include at least one of carbamyl phosphate, acetyl phosphate, propionyl phosphate, and butyryl phosphate, phosphono-acetic acid. See, e.g., Hosain et al. (1978) “Bone accumulation of the Tc-99m complex of carbamyl phosphate and its analogs,” J. Nucl. Med. 19:530-533, which is incorporated herein by reference.

In an aspect, the bone-targeting agent 120 includes calcium. In an aspect, the bone-targeting agent includes members of the IIA family of the periodic table which carry the same divalent charge as elemental calcium and are incorporated into bone matrix directly. For example, the bone-targeting agent can include strontium. For example, the bone-targeting agent can include radium.

In an aspect, the bone-targeting agent 120 comprises a bone morphogenetic protein. For example, the bone-targeting agent can include any of a number of bone morphogenetic proteins known to induce formation of bone and/or cartilage. In an aspect, the bone morphogenetic protein includes BMP2 or BMP4. In an aspect, the bone morphogenetic protein includes BMP7. In an aspect, the bone-targeting agent includes a recombinant form of a bone morphogenetic protein. For example, the bone-targeting agent can include recombinant human BMP2 (rhBMP2) or recombinant human BMP7 (rhBMP7). Non-limiting examples of bone morphogenetic proteins include BMP1, BMP2, BMP3, BMP4, BMP5, BMP6, BMP7, BMP8a, BMP8b, BMP10, and BMP15. See, e.g., Ducy & Karsenty (2000) “The family of bone morphogenetic proteins,” Kidney International 57:2207-2214; Granjeiro et al. (2005) “Bone morphogenetic proteins: from structure to clinical use,” Braz. J. Med. Biol. Res. 38:1463-1473, which are incorporated herein by reference.

In an aspect, the bone-targeting agent 120 comprises a hydroxyapatite-binding polypeptide 340. In an aspect, the bone-targeting agent includes negatively charged calcium-binding domains. For example, a hydroxyapatite-binding polypeptide can include a plurality of aspartic acid moieties (polyaspartate). In an aspect, a hydroxyapatite-binding polypeptide includes a plurality of glutamic acids (polyglutamate). For example, a string of aspartic acids (poly(aspartic acid)) can be conjugated to an inhibitor of nitric oxide synthase uncoupling to confer bone-targeting, bone-seeking, or osteotrophic properties to the complex. Other non-limiting examples of hydroxyapatite-binding polypeptides are described in U.S. Pat. No. 8,022,040 to Bertozzi et al. titled “Hydroxyapatite-binding peptides for bone growth and inhibition,” which is incorporated herein by reference.

Bone-targeting complex 100 includes linker 130. In an aspect, linker 130 includes a peptidyl linker of two or more amino acids. In an aspect, linker 130 includes an oligonucleotide or oligomer of two or more nucleotides. In an aspect, linker 130 includes a ligand/receptor pair. In an aspect, linker 130 includes an oligosaccharide. In an aspect, linker 130 includes an acyl chain. In general, the linker is configured to couple the inhibitor of nitric oxide synthase uncoupling to the bone-targeting agent.

In an embodiment, the inhibitor of nitric oxide synthase uncoupling 110 is coupled to a first end of the linker 130 and the bone-targeting agent 120 is coupled to a second end of the linker 130. For example, the inhibitor of nitric oxide synthase uncoupling and the bone-targeting agent can be respectively coupled to the first and the second end of the linker through non-covalent bonding, e.g., through ionic, hydrogen, or halogen bonding and/or Van der Waals forces, π effects, or hydrophobic interactions. For example, the inhibitor of nitric oxide synthase uncoupling and the bone-targeting agent can be respectively coupled to the first and the second end of the linker through a covalent or chemical bond. In an embodiment, the inhibitor of nitric oxide synthase uncoupling 110 is conjugated to a first end of the linker 130 and the bone-targeting agent 120 is conjugated to a second end of the linker 130.

In an aspect, linker 130 is configured to link an inhibitor of nitric oxide synthase uncoupling 110 to a bone-targeting agent 120. In an aspect, the linker comprises a disulfide linker, a carbamate linker, an amide linker, an ester linker, or an ether linker. In an aspect, the linker includes a chemical crosslinker. In an aspect, the chemical crosslinker includes an amine-reactive crosslinker. For example, the amine reactive crosslinker can include at least one of an imidoester crosslinker or an N-hydroxysuccinimide-ester crosslinker. In an aspect, the chemical crosslinker includes a sulfhydryl-reactive crosslinker. For example, the sulfhydryl-reactive crosslinker can include a maleimide crosslinker or a haloacetyl crosslinker. In an aspect, the chemical crosslinker includes pyridyl disulfides for crosslinking sulfhydryl groups. In an aspect, the chemical crosslinker includes a carbonyl-/glycol-reactive crosslinker, e.g., a hydrazide crosslinker. In an aspect, the chemical crosslinker includes a carboxyl-reactive crosslinker, e.g., a carbodiimide crosslinker. In an aspect, the chemical crosslinker includes an aryl azide crosslinker. Numerous examples of chemical crosslinkers are commercially available from, e.g., Thermo Fisher Scientific, Waltham, Mass. Also see, e.g., “Thermo Scientific Pierce Crosslinking Technical Handbook” published by Thermo Fisher Scientific and incorporated herein by reference.

In an embodiment, the linker includes a ligand/receptor pair. For example, the linker can include a biotin/avidin pair, wherein the avidin is covalently attached to the inhibitor of nitric oxide synthase uncoupling and the biotin is covalently attached to the bone-targeting agent. Ligand/receptor pairs can include antigen/antibody, co-factor/protein, and substrate/enzyme pairs. Non-limiting examples include biotin/avidin, biotin/streptavidin, FK506/FK506-binding protein (FKBP), rapamycin/FKBP, cyclophilin/cyclosporine, and glutathione/glutathione transferase pairs.

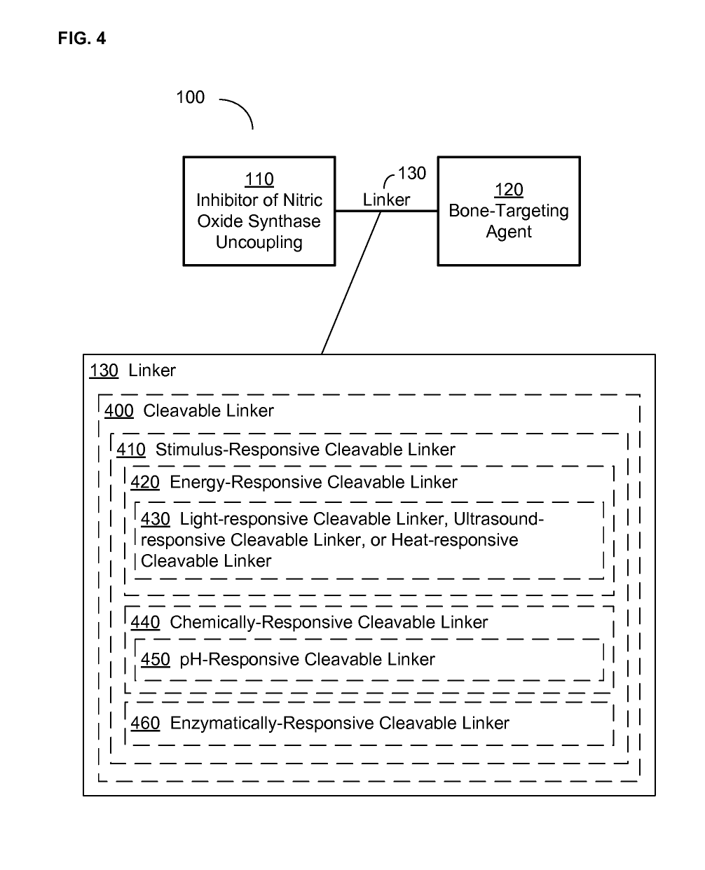

FIG. 4 illustrates further aspects of bone-targeting complex 100. FIG. 4 shows a block diagram of bone-targeting complex 100 including inhibitor of nitric oxide synthase uncoupling 110, bone-targeting agent 120, and linker 130. Also shown are non-limiting embodiments of linkers.

In an aspect, linker 130 comprises cleavable linker 400. For example, the linker can include a cleavable linker that is cleaved at some point after administration of the composition to a subject to release the inhibitor of nitric oxide synthase uncoupling from the bone-targeting agent. In an aspect, the cleavable linker is cleavable under extracellular conditions, releasing the inhibitor of nitric oxide synthase uncoupling from the bone-targeting agent in an extracellular environment. In an aspect, the cleavable linker is cleavable under intracellular conditions, releasing the inhibitor of nitric oxide synthase uncoupling from the bone-targeting agent in an intracellular environment. For example, the cleavable linker can be a peptidyl linker that is cleaved enzymatically by an intracellular peptidase or protease. For example, the cleavable linker can be cleaved in response to a pH change associated with an organelle, e.g., the lysosome, endosome, peroxisome, or caveolea.

In an aspect, cleavable linker 400 comprises a stimulus-responsive cleavable linker 410. For example, the cleavable linker can be responsive to an endogenous stimulus, e.g., a stimulus emanating from the subject to whom the composition has been administered. Non-limiting examples of stimuli emanating from the subject include pH changes, temperature changes, and enzymatic or other chemical activity. For example, the cleavable linker can be responsive to an exogenous stimulus, e.g., a stimulus emanating from outside the subject to whom the composition has been administered. Non-limiting examples of stimuli emanating from outside the subject include energy stimuli, e.g., light, ultrasound, or heat.

In an aspect, stimulus-responsive cleavable linker 410 comprises an energy-responsive cleavable linker 420. For example, the cleavable linker can be responsive to an energy stimulus. Non-limiting examples of energy stimuli include electromagnetic energy, acoustic energy, magnetic energy, light energy, radiofrequency energy, and/or microwave energy. In an aspect, the energy-responsive cleavable linker 420 comprises at least one of a light-responsive cleavable linker, an ultrasound-responsive cleavable linker, or heat-responsive cleavable linker 430.

In an aspect, the energy-responsive cleavable linker includes a light-responsive cleavable linker. For example, the light-responsive cleavable linker can include a photolabile linker responsive to ultraviolet, visible, and/or infrared light. In an aspect, the light-responsive cleavable linker includes a photolabile carboxylic acid, carboxamide, amidine, or hydroxyl group. In an aspect, ultraviolet and short visible (wavelength less than 400 nm) light are used to stimulate cleavage on or near the skin surface. For example, the light-responsive cleavable linker can include photocleavable 1-(2-nitrophenyl)ethyl phosphate esters or a photocleavable 2-nitrobenzyl group cleavable at ultraviolet wavelengths. See, e.g., U.S. Pat. No. 5,434,272 to Corrie & Trentham titled “Photo-labile compounds, their synthesis and use as fluorophores,” which is incorporated herein by reference. For example, the light-responsive cleavable linker can include the photolabile linker 4-{4-[1-(9-Fluorenylmethyloxycarbonylamino)ethyl]-2-methoxy-5-nitrophenoxy}butanoic acid from Advanced Chemtech, Louisville, Ky. or Novabiochem/EMD Millipore, Billerica, Mass. In an aspect, long visible and near infrared (wavelengths between 650 and 1000 nm) light are used to stimulate cleavage deeper into the tissue. See, e.g., Moses & You (2013) “Emerging strategies for controlling drug release by using visible/near IR light,” Med. Chem. 3:192-198, which is incorporated herein by reference.

In an aspect, the energy-responsive cleavable linker includes an ultrasound-responsive cleavable linker. For example, the linker coupling the inhibitor of nitric oxide synthase uncoupling to the bone-targeting agent can include an ultrasound-responsive cleavable linker configured to cleave in response to externally applied ultrasound energy. See, e.g., U.S. Patent Application 2012/0035531 from Zhao et al. titled “On-Demand and Reversible Drug Release by External Cue,” which is incorporated herein by reference.

In an aspect, the energy-responsive cleavable linker includes a heat-responsive cleavable linker. For example, the linker coupling the inhibitor of nitric oxide synthase uncoupling to the bone-targeting agent can include a heat-responsive cleavable linker configured to cleave in response to either internal heat changes associated with the subject or externally applied heat or thermal energy. See, e.g., U.S. Patent Application 2010/0068260 from Kruse et al. titled “Methods, Compositions, and Device for Directed and Controlled Heating and Release of Agents,” which is incorporated herein by reference. In an aspect, the heat-responsive cleavable linker is responsive to heat generated by the application of near-infrared radiation.

In an aspect, stimulus-responsive cleavable linker 410 comprises a chemically-responsive cleavable linker 440. For example, a bone-targeting complex can include a chemically-responsive cleavable linker coupling the inhibitor of nitric oxide synthase uncoupling to the bone-targeting agent that is responsive to a chemical reaction or condition. For example, the chemically-responsive cleavable linker can be configured to be responsive to oxidizing conditions, reducing conditions, and/or pH conditions. See, e.g., Amore et al. (2012) ChemBioChem 14:123-131; and U.S. Pat. No. 4,880,935 to Thorpe titled “Heterobifunctional linking agents derived from N-succinimido-dithio-alpha methyl-methylene-benzoates,” which are incorporated herein by reference. In an aspect, the chemically-responsive cleavable linker can include a superoxide cleavable linker (aminoacrylate) responsive to superoxide released in response to light exposure. See, e.g., U.S. Patent Application No. 2015/0165026 from You et al. titled “Singlet oxygen-labile linkers and methods of production and use thereof,” which is incorporated herein by reference.

In an aspect, the chemically-responsive cleavable linker 440 comprises a pH-responsive cleavable linker 450. For example, the cleavable linker can include a pH-sensitive cleavable linker, e.g., sensitive to hydrolysis/cleavage at certain pH values. For example, the pH-responsive cleavable linker can be responsive to changes in pH as the composition is brought into a cell or into a subcellular organelle, e.g., the lysosome. For example, the cleavable linker can include an acid-labile linker responsive to an acidic pH (e.g., an amino-sulfhydryl, thioether, hydrazone, semicarbazone, thiosemicarbazone, cis-aconitic amide, orthoester, acetal, ketal, or the like). See, e.g., U.S. Pat. No. 4,618,492 to Blattler et al. titled “Acid-cleavable compound;” U.S. Pat. No. 5,122,368 to Greenfield et al. titled “Anthracycline conjugates having a novel linker and methods for their production;” U.S. Pat. No. 5,824,805 to King et al. titled “Branched hydrazone linkers;” and U.S. Pat. No. 5,622,929 to Willner et al. titled “Thioether conjugates,” all of which are incorporated herein by reference.

In an embodiment, the chemically-responsive cleavable linker 440 includes a linker (e.g., a disulfide linker) cleavable under reducing conditions. Non-limiting examples of disulfide linkers include SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene) and are available from commercial sources (from, e.g., Thermo Fisher Scientific, Waltham, Mass.). Also see, e.g., U.S. Pat. No. 4,880,935 to Thorpe titled “Heterobifunctional linking agents derived from N-succinimido-dithio-alpha methyl-methylene-benzoates,” which is incorporated herein by reference.

In an aspect, the stimulus-responsive cleavable linker 410 comprises an enzymatically-responsive cleavable linker 460. For example, a bone-targeting complex can include a cleavable linker coupling the inhibitor of nitric oxide synthase uncoupling to the bone-targeting agent that is response to an enzymatic activity endogenous to the subject to whom the bone-targeting complex has been administered. For example, the cleavable linker can be responsive to an enzymatic activity specific to a target cell, e.g., a bone cell, to which the composition is directed or accumulates. For example, the enzymatically-responsive cleavable linker can include a peptidase- or protease-sensitive dipeptide or oligopeptide sensitive to cleavage by a peptidase or protease enzyme. In an aspect, the enzymatic stimulus, e.g., a peptidase or protease enzyme, is present in the intracellular environment, e.g., within a lysosome, endosome, or caveolea. In an aspect, the enzymatically-responsive cleavable linker is designed such that there is little or no cleavage of the linker in the plasma. In an aspect, the enzymatically-responsive cleavable linker is cleavable in response to a bone-specific peptidase or protease. For example, the enzymatically-responsive cleavable linker can include a peptide sequence cleavable by cathepsin K, a cysteine protease. See, e.g., Choi et al. (2012) “Protease-activated drug development,” Theranostics, 2:156-178, which is incorporated herein by reference. For example, the enzymatically-responsive cleavable linker can include a peptide sequence cleavable by one or more matrix metalloproteinases. See, e.g., U.S. Patent Application 2004/0116348 from Chau & Langer titled “Polymer-linker-drug conjugates for targeted drug delivery,” which is incorporated herein by reference. Additional non-limiting examples of enzymatically cleavable linkers are described in U.S. Pat. No. 6,214,345 to Firestone & Dubowchik titled “Lysosomal enzyme-cleavable antitumor drug conjugates;” and U.S. Pat. No. 8,968,742 to Morrison et al. titled “Antibody drug conjugates (ADC) that bind to 158P1D7 proteins,” which are incorporated herein by reference.

In some embodiments, a composition further includes at least one carrier or excipient mixed with the bone-targeting complex to form at least one of a topical dosage form, an enteral dosage form, or a parenteral dosage form for delivery to a subject. In an aspect, the dosage form is formulated for delivery to the subject by at least one of peroral delivery, oral delivery, topical delivery, transdermal delivery, epidermal delivery, intravitreal delivery, transmucosal delivery, inhalation, surgical delivery, or injection delivery. In an aspect, the dosage form includes at least one solid, liquid, or gas. In an aspect, the dosage form includes at least one of an aerosol, gel, sol, ointment, solution, suspension, capsule, tablet, cachets, suppository, cream, device, paste, liniment, lotion, ampule, elixir, emulsion, microemulsion, spray, suspension, powder, syrup, tincture, detection material, polymer, biopolymer, buffer, adjuvant, diluent, lubricant, disintegration agent, suspending agent, solvent, colorant, glidant, anti-adherent, anti-static agent, surfactant, plasticizer, emulsifying agent, flavor, gum, sweetener, coating, binder, filler, compression aid, encapsulation aid, preservative, granulation agent, spheronization agent, stabilizer, adhesive, pigment, sorbent, or nanoparticle.

The formulation of any of the compositions described herein may be formulated neat or may be combined with one or more acceptable carriers, diluents, excipients, and/or vehicles such as, for example, buffers, surfactants, preservatives, solubilizing agents, isotonicity agents, and stablilizing agents as appropriate. A “pharmaceutically acceptable” carrier, for example, may be approved by a regulatory agency of the state and/or Federal government such as, for example, the United States Food and Drug Administration (US FDA) or listed in the U.S. Pharmacopeia or other generally recognized pharmacopeia for use in animals, and more particularly in humans. Conventional formulation techniques generally known to practitioners are described in Remington: The Science and Practice of Pharmacy, 20^{th} Edition, Lippincott Williams & White, Baltimore, Md. (2000), which is herein incorporated by reference.

Acceptable excipients include, but are not limited to, the following: sugars, such as lactose, glucose and sucrose; starches, such as corn starch and potato starch; cellulose, and its derivatives, such as sodium carboxymethyl cellulose, ethyl cellulose, cellulose acetate, and hydroxymethylcellulose; polyvinylpyrrolidone; cyclodextrin and amylose; powdered tragacanth; malt; gelatin, agar and pectin; talc; oils, such as mineral oil, polyhydroxyethoxylated castor oil, peanut oil, cottonseed oil, safflower oil, sesame oil, olive oil, corn oil and soybean oil; polysaccharides, such as alginic acid and acacia; fatty acids and fatty acid derivatives, such as stearic acid, magnesium and sodium stearate, fatty acid amines, pentaerythritol fatty acid esters; and fatty acid monoglycerides and diglycerides; glycols, such as propylene glycol; polyols, such as glycerin, sorbitol, mannitol and polyethylene glycol; esters, such as ethyl oleate and ethyl laurate; buffering agents, such as magnesium hydroxide, aluminum hydroxide and sodium benzoate/benzoic acid; water; isotonic saline; Ringer's solution; ethyl alcohol; phosphate buffer solutions; other non-toxic compatible substances employed in pharmaceutical compositions. The compositions are generally formulated as sterile, substantially isotonic and in full compliance with all Good Manufacturing Practice (GMP) regulations of the U.S. Food and Drug Administration.

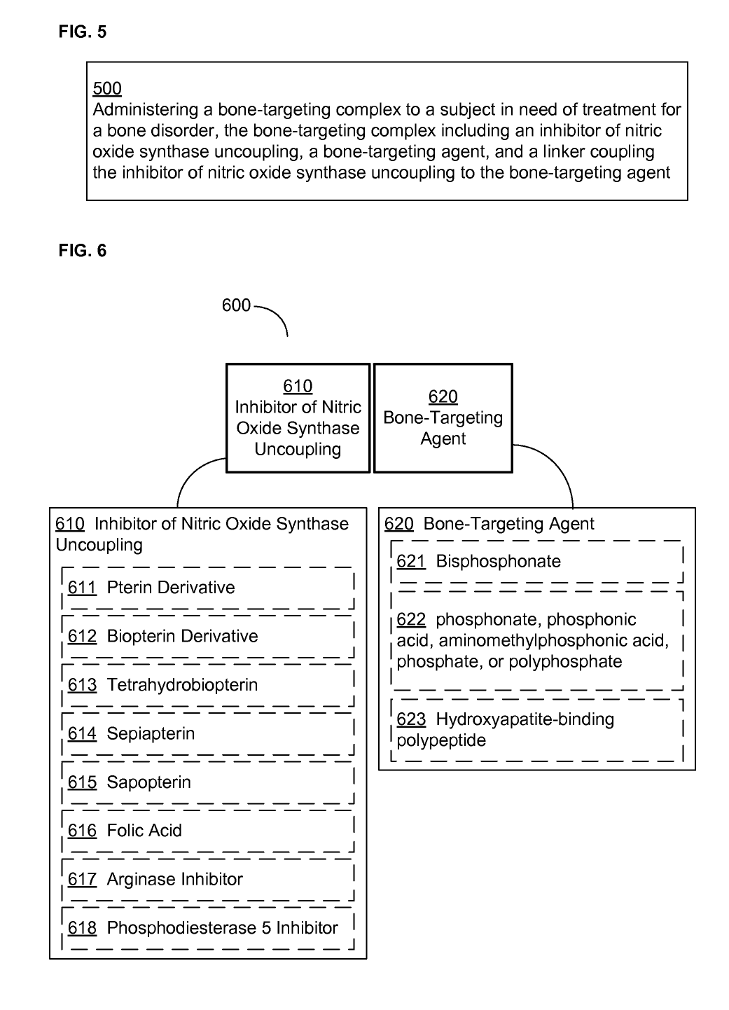

FIG. 5 illustrates a method of treating a bone disorder. In some embodiments, a method 500 of treating a bone disorder includes administering a bone-targeting complex to a subject in need of treatment for a bone disorder, the bone-targeting complex including an inhibitor of nitric oxide synthase uncoupling, a bone-targeting agent, and a linker coupling the inhibitor of nitric oxide synthase uncoupling to the bone-targeting agent. In an aspect, the method includes administering the bone-targeting complex to a human subject. In an aspect, the method includes administering the bone-targeting complex to a mammalian subject.

In an aspect, method 500 includes topically, enterally, or parenterally administering the bone-targeting complex to the subject in need of treatment for the bone disorder. For example, the method can include topically applying a composition including the bone-targeting complex to a skin surface in proximity to a bone and/or joint in need of treatment for a bone disorder, e.g., osteoarthritis or osteoporosis. For example, the method can include orally administering a liquid, tablet, or capsule including a composition including the bone-targeting complex to a subject in need of treatment for a bone disorder. For example, the method can include injecting a liquid composition including the bone-targeting complex into a subject in need of treatment for a bone disorder. In an embodiment, the method includes injecting a liquid composition including the bone-targeting complex directly into a bone region in need of treatment.

In an aspect, a method of treating a bone disorder includes administering a bone-targeting complex to a subject in need of treatment for a bone disorder, the bone-targeting complex including an inhibitor of nitric oxide synthase uncoupling, a bone-targeting agent, and a cleavable linker coupling the inhibitor of nitric oxide synthase uncoupling. In an aspect, the cleavable linker includes at least one of an energy-responsive cleavable linker, a chemically-responsive cleavable linker, or an enzymatically-responsive cleavable linker.

In some embodiments, a composition comprises a bone-targeting complex including an inhibitor of nitric oxide synthase uncoupling, and a bone-targeting agent associated with the inhibitor of nitric oxide synthase uncoupling.

In some embodiments, a bone-targeting complex includes an inhibitor of nitric oxide synthase uncoupling directly associated with a bone-targeting agent. FIG. 6 illustrates aspects of such a bone-targeting complex. FIG. 6 shows a block diagram of bone-targeting complex 600 including inhibitor of nitric oxide synthase uncoupling 610 and bone-targeting agent 620 associated with the inhibitor of nitric oxide synthase uncoupling 610.

Bone-targeting complex 600 includes inhibitor of nitric oxide synthase uncoupling 610. In some embodiments, the inhibitor of nitric oxide synthase uncoupling 610 is configured to or has the properties of preventing the uncoupling of nitric oxide synthase and as such prolonging the nitric oxide (NO) generating activity of the nitric oxide synthase. In some embodiments, the inhibitor of nitric oxide synthase uncoupling 610 is configured to or has the properties of restoring nitric oxide synthase to a coupled form and as such restoring the nitric oxide (NO) generating activity of the nitric oxide synthase. In an aspect, the inhibitor of nitric oxide synthase uncoupling comprises an inhibitor of endothelial nitric oxide synthase uncoupling. In an aspect, the inhibitor of nitric oxide synthase uncoupling comprises an inhibitor of neuronal nitric oxide synthase uncoupling. In an aspect, the inhibitor of nitric oxide synthase uncoupling comprises an inhibitor of inducible nitric oxide synthase uncoupling.

In an aspect, the inhibitor of nitric oxide synthase uncoupling 610 comprises pterin derivative 611. In an aspect, the inhibitor of nitric oxide synthase uncoupling 610 comprises biopterin derivative 612. In an aspect, the inhibitor of nitric oxide synthase uncoupling 610 comprises tetrahydrobiopterin 613. In an aspect, the inhibitor of nitric oxide synthase uncoupling 610 comprises sepiapterin 614. In an aspect, the inhibitor of nitric oxide synthase uncoupling 610 comprises sapopterin 615. In an aspect, the inhibitor of nitric oxide synthase uncoupling 610 comprises folic acid 616. In an aspect, the inhibitor of nitric oxide synthase uncoupling 610 comprises arginase inhibitor 617. In an aspect, the inhibitor of nitric oxide synthase uncoupling 610 comprises phosphodiesterase 5 inhibitor 618. Non-limiting examples of inhibitors of nitric oxide synthase uncoupling have been described above herein.

Bone-targeting complex 600 includes bone-targeting agent 620 associated with the inhibitor of nitric oxide synthase uncoupling 610. The bone-targeting agent 620 is configured to or has the properties of selectively accumulating in bone tissue and cells. In an aspect, the bone-targeting agent 620 includes an osteotropic agent. In an aspect, the bone-targeting agent 620 includes a “targetor” moiety able to recognize bones cells or components thereof. In an aspect, the bone-targeting agent 620 comprises bisphosphonate 621. In an aspect, the bone-targeting agent 620 includes a derivative of bisphosphonate. In an aspect, the bone-targeting agent 620 comprises at least one of phosphonate, phosphonic acid, aminomethylphosphonic acid, phosphate, or polyphosphate, as shown in block 622. In an aspect, the bone-targeting agent 620 comprises bone-morphogenetic protein (BMP). For example, the bone-targeting agent can include BMP2, BMP4, or BMP7. In an aspect, the bone-targeting agent 620 comprises hydroxyapatite-binding polypeptide 623. Non-limiting examples of bone-targeting agents have been described above herein.

In an embodiment, the inhibitor of nitric oxide synthase uncoupling 610 is non-covalently associated with the bone-targeting agent 620. For example, the inhibitor of nitric oxide synthase uncoupling and the bone-targeting agent can be respectively coupled to one another through non-covalent bonding, e.g., through ionic, hydrogen, or halogen bonding and/or Van der Waals forces, π effects, or hydrophobic interactions. In an embodiment, the inhibitor of nitric oxide synthase uncoupling 610 is covalently associated with the bone-targeting agent 620. For example, the inhibitor of nitric oxide synthase uncoupling and the bone-targeting agent can be coupled to one another through a covalent or chemical bond.

In some embodiments, the composition includes a linker coupling the inhibitor of nitric oxide synthase uncoupling 610 to the bone-targeting agent 620. In an aspect, the linker includes a first end coupled to the inhibitor of nitric oxide synthase uncoupling 610 and a second end coupled to the bone-targeting agent 620. In an aspect, the linker includes a first end conjugated to the inhibitor of nitric oxide synthase uncoupling 610 and a second end conjugated to the bone-targeting agent 620. In an aspect, the linker comprises a disulfide linker, a carbamate linker, an amide linker, an ester linker, or an ether linker. In an aspect, the linker includes a chemical crosslinker. In an aspect, the linker comprises a cleavable linker. In an aspect, the cleavable linker comprises a stimulus-responsive cleavable linker. In an aspect, the stimulus-responsive cleavable linker comprises at least one of an energy-responsive cleavable linker, a chemically-responsive cleavable linker, or an enzymatically-responsive cleavable linker Non-limiting aspects of linkers and cleavable linkers have been described above herein.

In an embodiment, the composition including bone-targeting complex 600 further at least one carrier or excipient mixed with the bone-targeting complex to form at least one of a topical dosage form, an enteral dosage form, or a parenteral dosage form for delivery to a subject. Non-limiting aspects of carrier, excipients, and formulations have been described above herein.

In some embodiments, a composition includes a bone-targeting complex including an activator of nitric oxide synthase, a bone-targeting agent, and a linker coupling the activator of nitric oxide synthase to the bone-targeting agent.

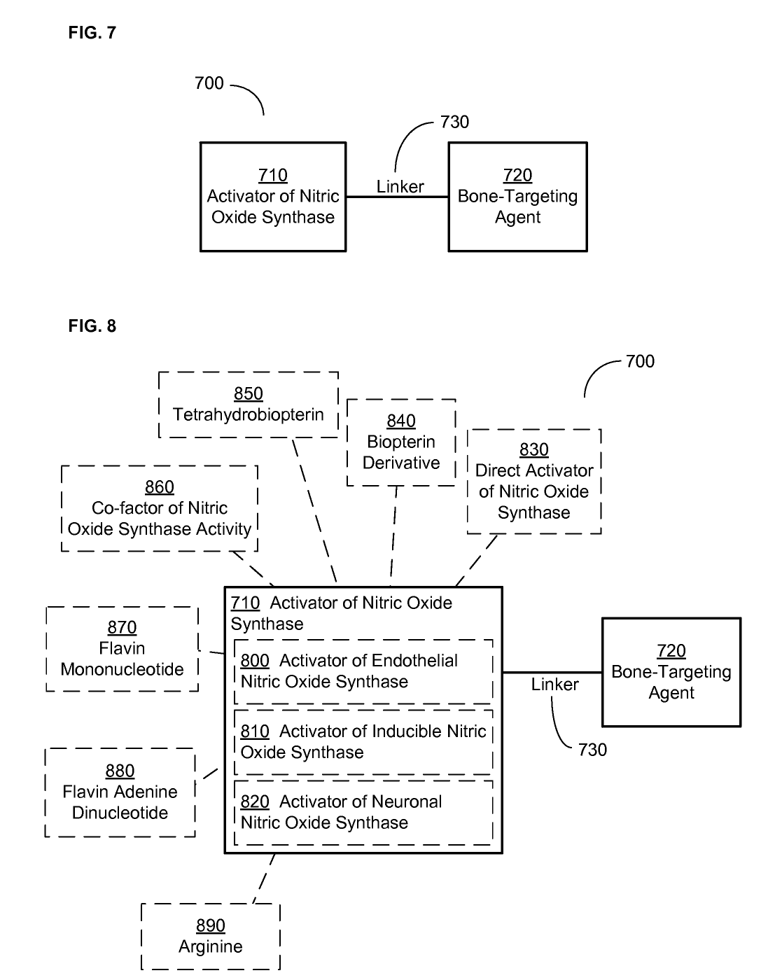

FIG. 7 illustrates aspects of a composition including a bone-targeting complex with an activator of nitric oxide synthase, a bone-targeting agent, and a linker. FIG. 7 shows a block diagram of bone-targeting complex 700 including activator of nitric oxide synthase 710, bone-targeting agent 720, and linker 730 coupling the activator of nitric oxide synthase 710 to the bone-targeting agent 720.

In some embodiments, a bone-targeting complex includes an activator of nitric oxide synthase. An activator of nitric oxide synthase includes an agent configured to or having the property of activating or enhancing the activity of nitric oxide synthase to increase the production of nitric oxide.

FIG. 8 is a block diagram illustrating further aspects of a composition including bone-targeting complex 700. Shown is bone-targeting complex 700 including an activator of nitric oxide synthase 710, bone-targeting agent 720, and linker 730. Also shown are non-limiting embodiments of an activator of nitric oxide synthase 710. In an aspect, the activator of nitric oxide synthase comprises an activator of endothelial nitric oxide synthase 800. In an aspect, the activator of nitric oxide synthase comprises an activator of inducible nitric oxide synthase 810. In an aspect, the activator of nitric oxide synthase comprises an activator of neuronal nitric oxide synthase 820.

In an embodiment, the activator of nitric oxide synthase 710 comprises a direct activator of nitric oxide synthase activity 830. For example, the activator of nitric oxide synthase can include a substrate (e.g., arginine) and/or co-factor (e.g., tetrahydrobiopterin) associated with activation and/or continued activity of nitric oxide synthase.

In an aspect, the activator of nitric oxide synthase 710 comprises a biopterin derivative 840. Non-limiting examples of biopterin derivatives have been described above herein. In an aspect, the activator of nitric oxide synthase 710 comprises tetrahydrobiopterin 850. In an aspect, the activator of nitric oxide synthase 710 includes a derivative of tetrahydrobiopterin. Non-limiting examples of tetrahydrobiopterin derivatives have been described above herein.

In an aspect, the activator of nitric oxide synthase 710 comprises a co-factor of nitric oxide synthase 860. In some embodiments, the co-factor is necessary for activity of the nitric oxide synthase. In some embodiments, the co-factor accelerates the activity of the nitric oxide synthase. Non-limiting examples of co-factors of nitric oxide synthase include flavin adenine dinucleotide, flavin mononucleotide, heme, calmodulin, and tetrahydrobiopterin. For example, the bone-targeting complex can include calmodulin coupled to a bone-targeting agent through a linker. In an aspect, the activator of nitric oxide synthase 710 comprises flavin mononucleotide (FMN) 870. For example, the bone-targeting complex can include flavin mononucleotide coupled to a bone-targeting agent (e.g., bisphosphonate) through a linker. In an aspect, the activator of nitric oxide synthase 710 comprises flavin adenine dinucleotide (FAD) 880. For example, the bone-targeting complex can include flavin adenine dinucleotide coupled to a bone-targeting agent (e.g., bisphosphonate) through a linker. In an aspect, the activator of nitric oxide synthase includes riboflavin, the molecule from which both flavin mononucleotide and flavin adenine dinucleotide are derived. For example, the bone-targeting complex can include riboflavin coupled to a bone-targeting agent (e.g., bisphosphonate) through a linker.

In an aspect, the activator of nitric oxide synthase 710 comprises arginine 890. For example, the bone-targeting complex can include arginine coupled to a bone-targeting agent (e.g., a bisphosphonate derivative) through a linker.

In an embodiment, the activator of nitric oxide synthase is a precursor to a co-factor of nitric oxide synthase. For example, the activator of nitric oxide synthase can include riboflavin, a precursor to both flavin mononucleotide and flavin adenine dinucleotide. In an embodiment, the activator of nitric oxide synthase includes a co-factor mimetic. For example, the activator of nitric oxide synthase can include a chemical agent that mimics the effects of one or more of the co-factors of nitric oxide synthase activity. In an embodiment, the activator of nitric oxide synthase can include an agent that activates nitric oxide synthase by antagonizing autoinhibition of the enzyme. For example, the activator of nitric oxide synthase can include a peptide that binds to that portion of nitric oxide synthase involved in autoinhibition. See, e.g., U.S. Pat. No. 6,150,500 to Salerno titled “Activators of Endothelial Nitric Oxide Synthase,” which is incorporated herein by reference.

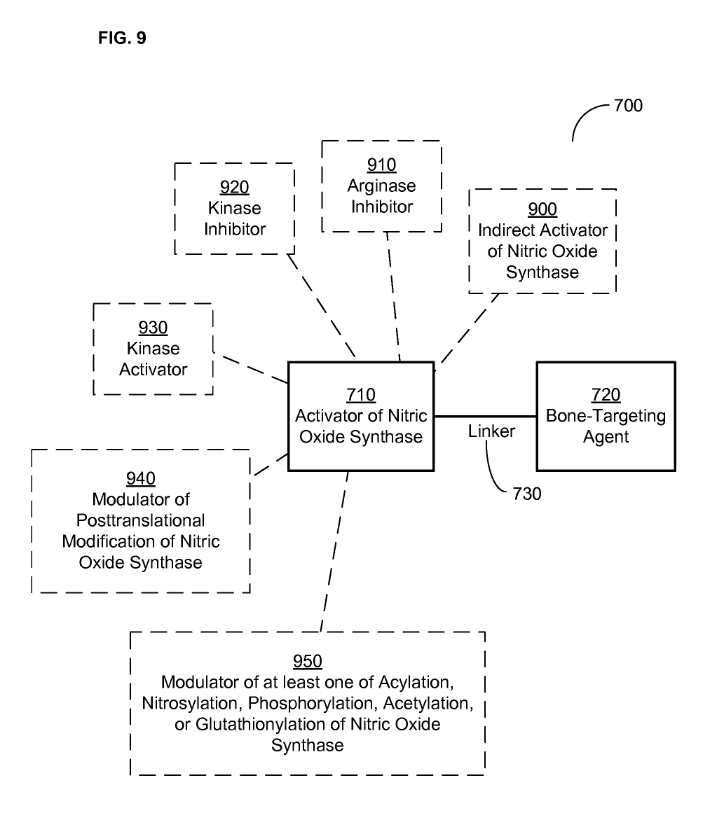

FIG. 9 illustrates further aspects of a composition including bone-targeting complex 700. In some embodiments, the activator of nitric oxide synthase comprises an indirect activator of nitric oxide synthase activity 900. For example, the activator of nitric oxide synthase can include an agonist or antagonist of another molecular entity, e.g., an enzyme, which modulates the activity of nitric oxide synthase. For example, the nitric oxide synthase can include an agonist or antagonist of a kinase capable of modulating the activity of nitric oxide synthase through phosphorylation events.

In an aspect, the activator of nitric oxide synthase 710 comprises an arginase inhibitor 910. For example, inhibition of arginase increases the availability of arginine as a substrate for nitric oxide synthase and inhibits, prevents, and/or reverses uncoupling of nitric oxide synthase activity. In an aspect, the arginase inhibitor includes ornithine. See, e.g., U.S. Pat. No. 5,767,160 to Kaesemeyer titled “Method and Formulation of Stimulating Nitric Oxide Synthesis,” which is incorporated herein by reference. In an aspect, the arginase inhibitor includes N-hydroxy-guanidinium derivatives; boronic acid derivatives (e.g., 2(S)-amino-6-boronohexanoic acid and S-(2-boronoethyl)-1-cysteine (BEC)), and (R)-2-amino-6-borono-2-(2-(piperidin-1-yl)ethyl)hexanoic acid. See, e.g., Steppan et al. (2013) “Development of novel arginase inhibitors for therapy of endothelial dysfunction,” Front. Immunol., September 17; 4:278. doi: 10.3389/fimmu.2013.00278, which is incorporated herein by reference. Additional non-limiting examples of inhibitors of arginase activity are described in U.S. Pat. No. 6,387,890 to Christianson et al. titled “Compositions and Methods for Inhibiting Arginase Activity;” and in U.S. Pat. No. 6,723,710 to Christianson et al. titled “Compositions for Inhibiting Arginase Activity,” which are incorporated herein by reference. Other non-limiting examples of arginase inhibitors include N^{ω}-hydroxy-nor-L-arginine (nor-NOHA) and N^{ω}-hydroxy-L-arginine (NOHA).

In an aspect, the activator of nitric oxide synthase 710 comprises a kinase inhibitor 920. For example, the activator of nitric oxide synthase can include an inhibitor or antagonist of a kinase responsible for phosphorylating nitric oxide synthase. In an aspect, the activator of nitric oxide synthase 710 comprises a kinase activator 930. For example, the activator of nitric oxide synthase can include an activator or agonist of a kinase responsible for phosphorylating nitric oxide synthase. For example, the activator of nitric oxide synthase can include an agonist or antagonist of at least one of Akt/protein kinase B (Akt), protein kinase A (PKA), adenosine monophosphate-activated protein kinase (AMPK), protein kinase G, CaMKII, and protein kinase C, kinases known to phosphorylate nitric oxide synthase. See, e.g., Heiss & Dirsch (2014) “Regulation of eNOS enzyme activity by posttranslational modification,” Curr. Pharm. Des. 20:3503-3513, which is incorporated by reference herein.

In an aspect, the activator of nitric oxide synthase 710 comprises a modulator of posttranslational modification of nitric oxide synthase 940. For example, the modulator of posttranslational modification of nitric oxide synthase can include an agonist or an antagonist of posttranslational modification of nitric oxide synthase. In an aspect, the activator of nitric oxide synthase comprises a modulator of at least one of acylation, nitrosylation, phosphorylation, acetylation, or glutathionylation of nitric oxide synthase 950. For example, the activator of nitric oxide synthase can include an agonist or antagonist of a phosphatase or kinase responsible for modulating the phosphorylation state of nitric oxide synthase, as previously described above herein. For example, the activator of nitric oxide synthase can include an activator of acylation of nitric oxide synthase as myristolylation and palmitoylation are required to target nitric oxide synthase to caveolae for optimal activity. See, e.g., Shaul et al. (1996) “Acylation targets endothelial nitric oxide synthase to plasmalemmal caveolae,” J. Biol. Chem., 271:6518-6522, which is incorporated herein by reference. For example, the activator of nitric oxide synthase can include an inhibitor of S-nitrosylation of nitric oxide synthase as the addition of NO to nitric oxide synthase has a negative feedback effect on the activity of nitric oxide synthase. See, e.g., Ravi et al. (2004) “S-nitrosylation of endothelial nitric oxide synthase is associated with monomerization and decreased enzyme activity,” Proc. Natl. Acad. Sci. USA, 101:2619-2624, which is incorporated herein by reference. For example, the activator of nitric oxide synthase can include an activator of acetylation of nitric oxide synthase by calreticulin transacetylase, as acetylation of nitric oxide synthase increases nitric oxide synthase activity. See, e.g., Ponnan et al. (2014) “Comparison of protein acetyltransferase action of CRTAase with the prototypes of HAT,” ScientificWorldJournal February 4; 2014:578956. doi: 10.1155/2014/578956, which is incorporated herein by reference. For example, the activator of nitric oxide synthase can include an inhibitor of S-glutathionylation of nitric oxide synthase, as S-glutathionylation of nitric oxide synthase attenuates the nitric oxide producing capability of nitric oxide synthase. See, e.g., Chen et al. (2010) “S-glutathionylation uncouples eNOS and regulates its cellular and vascular function,” Nature 468:1115-1118, which is incorporated herein by reference.

Bone-targeting complex 700 includes a bone-targeting agent 720. The bone-targeting agent is configured to or has the properties of selectively accumulating in bone tissue and cells. In an aspect, the bone-targeting agent includes an osteotropic agent. In an aspect, the bone-targeting agent includes a “targetor” moiety able to recognize bones cells or components thereof.

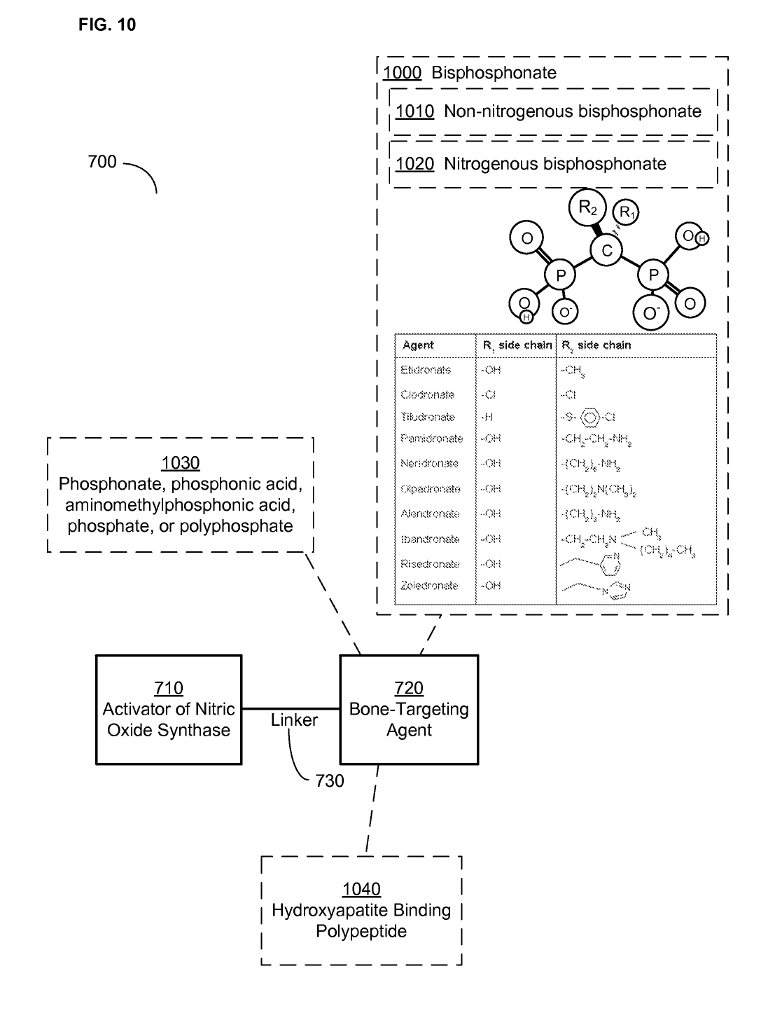

FIG. 10 illustrates further aspects of bone-targeting complex 700. FIG. 10 shows a block diagram of bone-targeting complex 700 including activator of nitric oxide synthase 710, bone-targeting agent 720, and linker 730. Also shown are non-limiting embodiments of bone-targeting agents.

In an aspect, the bone-targeting agent 720 comprises bisphosphonate 1000. In an aspect, bone-targeting agent 720 comprises a bisphosphonate derivative. In an aspect, bone-targeting agent 720 comprises a non-nitrogenous bisphosphonate 1010. Non-limiting examples of non-nitrogenous or non-nitrogen-containing bisphosphonates include etidronate, clodronate, and tiludronate. In an aspect, bone-targeting agent 720 comprises a nitrogenous bisphosphonate 1020. Non-limiting examples of nitrogenous bisphosphonates include pamidronate, neridronate, olpadronate, alendronate, ibandronate, risedronate, and zoledronate. Other non-limiting aspects of bisphosphonates have been described above herein.

In an aspect, the bone-targeting agent 720 includes an organic phosphate. In an aspect, the bone-targeting agent 720 comprises phosphonate, phosphonic acid, aminomethylphosphonic acid, phosphate, or polyphosphate, as shown in block 1030. In an aspect, the bone-targeting agent includes sodium orthophosphate or hydroxyethylidene diphosphonate. In an aspect, the bone-targeting agent includes a phosphate derivative. For example, the bone-targeting agent can include at least one of carbamyl phosphate, acetyl phosphate, propionyl phosphate, and butyryl phosphate, phosphono-acetic acid.

In an aspect, the bone-targeting agent 720 includes calcium. In an aspect, the bone-targeting agent includes members of the IIA family of the periodic table which carry the same divalent charge as elemental calcium and are incorporated into bone matrix directly. For example, the bone-targeting agent can include strontium. For example, the bone-targeting agent can include radium.

In an aspect, the bone-targeting agent 720 comprises a bone morphogenetic protein (BMP). For example, the bone-targeting agent can include any of a number of bone morphogenetic proteins known to induce formation of bone and/or cartilage. In an aspect, the bone morphogenetic protein comprises BMP2 or BMP4. In an aspect, the bone morphogenetic protein comprises BMP7. In an aspect, the bone-targeting agent 720 includes a recombinant form of a bone morphogenetic protein. For example, the bone-targeting agent can include recombinant human BMP2 (rhBMP2) or recombinant human BMP7 (rhBMP7). Non-limiting examples of bone morphogenetic proteins include BMP1, BMP2, BMP3, BMP4, BMP5, BMP6, BMP7, BMP8a, BMP8b, BMP10, and BMP15.

In an aspect, the bone-targeting agent 720 comprises a hydroxyapatite-binding polypeptide 1040. In an aspect, the bone-targeting agent includes negatively charged calcium-binding domains. For example, a hydroxyapatite-binding polypeptide can include a plurality of aspartic acid moieties (polyaspartate). In an aspect, a hydroxyapatite-binding polypeptide includes a plurality of glutamic acids (polyglutamate). For example, a string of aspartic acids (poly(aspartic acid)) can be conjugated to an inhibitor of nitric oxide synthase uncoupling to confer bone-targeting, bone-seeking, or osteotrophic properties to the complex. Other non-limiting examples of hydroxyapatite-binding polypeptides are described in U.S. Pat. No. 8,022,040 to Bertozzi et al. titled “Hydroxyapatite-binding peptides for bone growth and inhibition,” which is incorporated herein by reference.

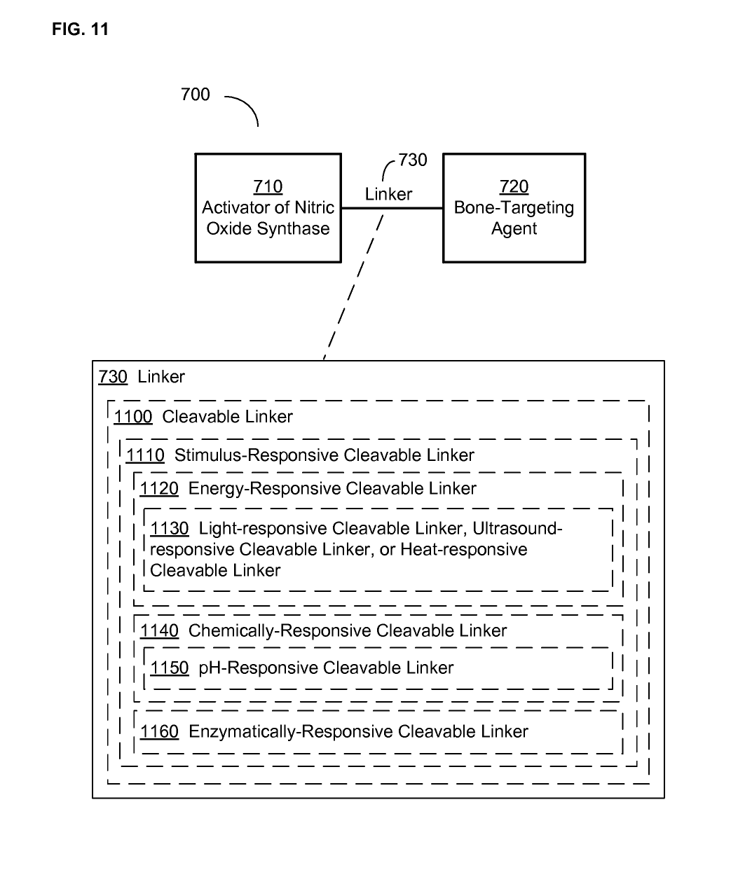

FIG. 11 illustrates further aspects of bone-targeting complex 700. FIG. 11 shows a block diagram of bone-targeting complex 700 including activator of nitric oxide synthase 710, bone-targeting agent 720, and linker 730. Also shown are non-limiting embodiments of linker 730.

In an aspect, linker 730 includes a peptidyl linker of two or more amino acids. In an aspect, linker 730 includes a ligand/receptor pair. In an aspect, linker 730 includes an oligonucleotide or oligomer of two or more nucleotides. In an aspect, linker 730 includes an oligosaccharide. In an aspect, linker 730 includes an acyl chain. In general, the linker is configured to couple the activator of nitric oxide synthase to the bone-targeting agent.

In an embodiment, the activator of nitric oxide synthase 710 is coupled to a first end of the linker 730 and the bone-targeting agent 720 is coupled to a second end of the linker 730. For example, the activator of nitric oxide synthase and the bone-targeting agent can be respectively coupled to the first and the second end of the linker through non-covalent bonding, e.g., through ionic, hydrogen, or halogen bonding and/or Van der Waals forces, π effects, or hydrophobic interactions. For example, the activator of nitric oxide synthase and the bone-targeting agent can be respectively coupled to the first and the second end of the linker through a covalent or chemical bond. In an embodiment, the activator of nitric oxide synthase 710 is conjugated to a first end of the linker 730 and the bone-targeting agent 720 is conjugated to a second end of the linker 730.

In an aspect, linker 730 is configured to link an activator of nitric oxide synthase 710 to a bone-targeting agent 720. In an aspect, linker 730 comprises a disulfide linker, a carbamate linker, an amide linker, an ester linker, or an ether linker. In an aspect, the linker includes a chemical crosslinker. Non-limiting examples of chemical crosslinkers have been described above herein. Numerous examples of chemical crosslinkers are commercially available from, e.g., Thermo Fisher Scientific, Waltham, Mass. Also see, e.g., “Thermo Scientific Pierce Crosslinking Technical Handbook” published by Thermo Fisher Scientific and incorporated herein by reference.

In an embodiment, linker 730 includes a ligand/receptor pair. For example, the linker can include a biotin/avidin pair, wherein the avidin is covalently attached to the activator of nitric oxide synthase and the biotin is covalently attached to the bone-targeting agent. Ligand/receptor pairs can include antigen/antibody, co-factor/protein, and substrate/enzyme pairs. Non-limiting examples of ligand/receptor pairs include biotin/avidin, biotin/streptavidin, FK506/FK506-binding protein (FKBP), rapamycin/FKBP, cyclophilin/cyclosporine, and glutathione/glutathione transferase pairs.

In an aspect, linker 730 comprises cleavable linker 1100. For example, the linker can include a cleavable linker that is cleaved at some point after administration of the composition to a subject to release the activator of nitric oxide synthase from the bone-targeting agent. In an aspect, cleavable linker 1100 is cleavable under extracellular conditions, releasing the activator of nitric oxide synthase from the bone-targeting agent in an extracellular environment. In an aspect, cleavable linker 1100 is cleavable under intracellular conditions, releasing the activator of nitric oxide synthase from the bone-targeting agent in an intracellular environment. For example, the cleavable linker can be a peptidyl linker that is cleaved enzymatically by an intracellular peptidase or protease. For example, the cleavable linker can be cleaved in response to a pH change associated with an organelle, e.g., the lysosome, endosome, peroxisome, or caveolea.

In an aspect, cleavable linker 1100 comprises a stimulus-responsive cleavable linker 1110. For example, the cleavable linker can be responsive to an endogenous stimulus, e.g., a stimulus emanating from the subject to whom the composition has been administered. Non-limiting examples of stimuli emanating from the subject include pH changes, tissue or cellular temperature changes, and enzymatic or other chemical activity. For example, the cleavable linker can be responsive to an exogenous stimulus, e.g., a stimulus emanating from outside the subject to whom the composition has been administered. Non-limiting examples of stimuli emanating from outside the subject include energy stimuli, e.g., light, ultrasound, or heat.

In an aspect, stimulus-responsive cleavable linker 1110 comprises an energy-responsive cleavable linker 1120. For example, the cleavable linker can be responsive to an energy stimulus. Non-limiting examples of energy stimuli include electromagnetic energy, acoustic energy, magnetic energy, light energy, radiofrequency energy, and/or microwave energy. In an aspect, the energy-responsive cleavable linker 1120 includes at least one of a light-responsive cleavable linker, an ultrasound-responsive cleavable linker, or a heat-responsive cleavable linker 1130. In an aspect, the energy-responsive cleavable linker 1120 includes a light-responsive cleavable linker. In an aspect, the energy-responsive cleavable linker 1120 includes an ultrasound-responsive cleavable linker. In an aspect, the energy-responsive cleavable linker 1120 includes a heat-responsive cleavable linker. Non-limiting examples of light-responsive, ultrasound-responsive, and heat-responsive cleavable linkers have been described above herein.

In an aspect, stimulus-responsive cleavable linker 1110 comprises a chemically-responsive cleavable linker 1140. For example, the stimulus-responsive cleavable linker can be responsive to a chemical reaction or condition. For example, the chemically-responsive cleavable linker can be configured to be responsive to oxidizing conditions, reducing conditions, and/or pH conditions. In an aspect, the chemically-responsive cleavable linker 1140 comprises a pH-responsive cleavable linker 1150. For example, the cleavable linker can include a pH-sensitive cleavable linker, e.g., sensitive to hydrolysis/cleavage at certain pH values. For example, the pH-responsive cleavable linker can be responsive to changes in pH as the composition is brought into a cell or into a subcellular organelle, e.g., the lysosome. For example, the cleavable linker can include an acid-labile linker responsive to an acidic pH (e.g., an amino-sulfhydryl, thioether, hydrazone, semicarbazone, thiosemicarbazone, cis-aconitic amide, orthoester, acetal, ketal, or the like).

In an aspect, the chemically-responsive cleavable linker 1140 is responsive to oxidation. For example, the chemically-responsive cleavable linker can be cleavable in response to the presence of superoxide. In an aspect, the chemically-responsive cleavable linker 1140 can include a disulfide group and be responsive to reducing conditions. Non-limiting examples of chemically-responsive cleavable linkers have been described above herein.

In an aspect, the stimulus-responsive cleavable linker 1110 comprises an enzymatically-responsive cleavable linker 1160. For example, the cleavable linker can be responsive to an enzymatic activity endogenous to the subject to whom the composition has been administered. For example, the cleavable linker can be responsive to an enzymatic activity specific to a target cell, e.g., a bone cell, to which the composition is directed or accumulates. For example, the enzymatically-responsive cleavable linker can include a peptidase- or protease-sensitive dipeptide or oligopeptide sensitive to cleavage by a peptidase or protease enzyme. In an aspect, the enzymatic stimulus, e.g., a peptidase or protease enzyme, is present in the intracellular environment, e.g., within a lysosome, endosome, or caveolea. In an aspect, the enzymatically-responsive cleavable linker is designed such that there is little or no cleavage of the linker in the plasma. In an aspect, the enzymatically-responsive cleavable linker is cleavable in response to a bone-specific peptidase or protease. For example, the enzymatically-responsive cleavable linker can include a peptide sequence cleavable by cathepsin K, a cysteine protease. For example, the enzymatically-responsive cleavable linker can include a peptide sequence cleavable by one or more matrix metalloproteinases. Non-limiting aspects of enzymatically-responsive cleavable linkers have been described above herein.

In an embodiment, the composition including bone-targeting complex 700 further includes at least one carrier or excipient mixed with the bone-targeting complex to form at least one of a topical dosage form, an enteral dosage form, or a parenteral dosage form for delivery to a subject. Non-limiting aspects of carrier, excipients, and formulations have been described above herein.

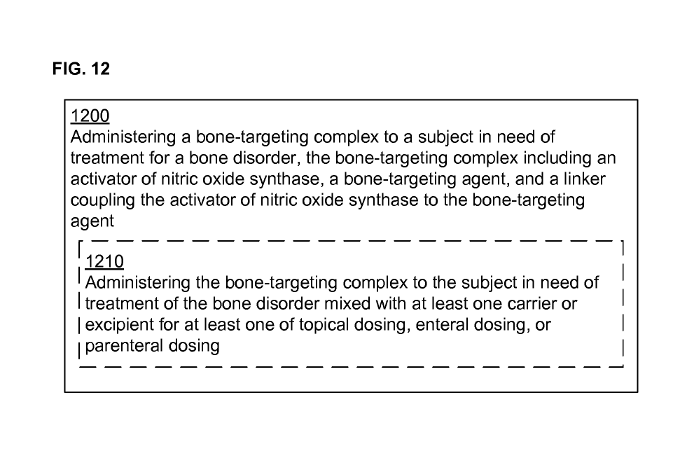

FIG. 12 is a block diagram illustrating a method of treating a bone disorder. In an embodiment, a method 1200 of treating a bone disorder includes administering a bone-targeting complex to a subject in need of treatment for a bone disorder, the bone-targeting complex including an activator of nitric oxide synthase, a bone-targeting agent, and a linker coupling the activator of nitric oxide synthase to the bone-targeting agent. In an aspect, the method 1200 further includes administering the bone-targeting complex to the subject in need of treatment for the bone disorder mixed with at least one carrier or excipient for at least one of topical dosing, enteral dosing, or parenteral dosing, as shown in block 1210.

In some embodiments, a composition comprises a bone-targeting complex including an activator of nitric oxide synthase and a bone-targeting agent associated with the activator of nitric oxide synthase.

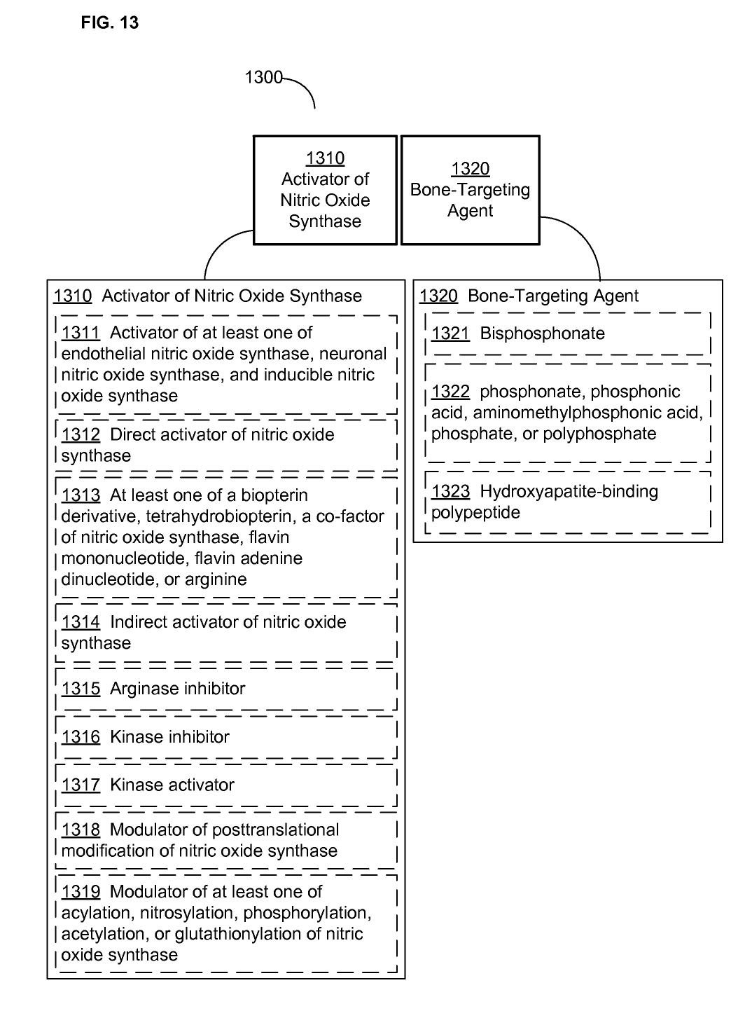

In some embodiments, a bone-targeting complex includes an activator of nitric oxide synthase directly associated with a bone-targeting agent. FIG. 13 illustrates aspects of such a bone-targeting complex. FIG. 13 shows a block diagram of bone-targeting complex 1300 including activator of nitric oxide synthase 1310 and bone-targeting agent 1320 associated with the activator of nitric oxide synthase 1310.

Bone-targeting complex 1300 includes an activator of nitric oxide synthase 1310. An activator of nitric oxide synthase includes an agent configured to or having the property of activating or enhancing the activity of nitric oxide synthase to increase the production of nitric oxide.

In an aspect, the activator of nitric oxide synthase 1310 comprises an activator of at least one of endothelial nitric oxide synthase, neuronal nitric oxide synthase, and inducible nitric oxide synthase, as shown in block 1311. In an embodiment, the activator of nitric oxide synthase 1310 comprises a direct activator of nitric oxide synthase activity 1312. For example, the activator of nitric oxide synthase can include a substrate (e.g., arginine) and/or co-factor (e.g., tetrahydrobiopterin) associated with activation and/or continued activity of nitric oxide synthase. In an aspect, the activator of nitric oxide synthase includes at least one of a biopterin derivative, tetrahydrobiopterin, a co-factor of nitric oxide synthase, flavin mononucleotide, flavin adenine dinucleotide, or arginine, as shown in block 1313. For example, the bone-targeting complex can include arginine coupled to a bone-targeting agent (e.g., a bisphosphonate derivative) through a linker.

In an aspect, the activator of nitric oxide synthase 1310 includes a co-factor necessary for activity of the nitric oxide synthase. In some embodiments, the co-factor accelerates the activity of the nitric oxide synthase. Non-limiting examples of co-factors of nitric oxide synthase include flavin adenine dinucleotide, flavin mononucleotide, heme, calmodulin, and tetrahydrobiopterin. For example, the bone-targeting complex can include calmodulin coupled to a bone-targeting agent through a linker. For example, the bone-targeting complex can include flavin mononucleotide coupled to a bone-targeting agent (e.g., bisphosphonate) through a linker. For example, the bone-targeting complex can include flavin adenine dinucleotide coupled to a bone-targeting agent (e.g., bisphosphonate) through a linker. In an aspect, the activator of nitric oxide synthase includes riboflavin, the molecule from which both flavin mononucleotide and flavin adenine dinucleotide are derived. For example, the bone-targeting complex can include riboflavin coupled to a bone-targeting agent (e.g., bisphosphonate) through a linker.

In an embodiment, the activator of nitric oxide synthase is a precursor to a co-factor of nitric oxide synthase. For example, the activator of nitric oxide synthase can include riboflavin, a precursor to both flavin mononucleotide and flavin adenine dinucleotide. In an embodiment, the activator of nitric oxide synthase includes a co-factor mimetic. For example, the activator of nitric oxide synthase can include a chemical agent that mimics the effects of one or more of the co-factors of nitric oxide synthase activity. In an embodiment, the activator of nitric oxide synthase can include an agent that activates nitric oxide synthase by antagonizing autoinhibition of the enzyme. For example, the activator of nitric oxide synthase can include a peptide that binds to that portion of nitric oxide synthase involved in autoinhibition. See, e.g., U.S. Pat. No. 6,150,500 to Salerno titled “Activators of Endothelial Nitric Oxide Synthase,” which is incorporated herein by reference.

In some embodiments, the activator of nitric oxide synthase comprises an indirect activator of nitric oxide synthase activity 1314. For example, the activator of nitric oxide synthase can include an agonist or antagonist of another molecular entity, e.g., an enzyme, which modulates the activity of nitric oxide synthase. For example, the nitric oxide synthase can include an agonist or antagonist of a kinase capable of modulating the activity of nitric oxide synthase through phosphorylation events.

In an aspect, the activator of nitric oxide synthase 1310 comprises an arginase inhibitor 1315. For example, inhibition of arginase increases the availability of arginine as a substrate for nitric oxide synthase and inhibits, prevents, and/or reverses uncoupling of nitric oxide synthase activity. In an aspect, the arginase inhibitor includes ornithine. Other non-limiting examples of arginase inhibitors have been described above herein.

In an aspect, the activator of nitric oxide synthase 1310 comprises a kinase inhibitor 1316. For example, the activator of nitric oxide synthase can include an inhibitor or antagonist of a kinase responsible for phosphorylating nitric oxide synthase. In an aspect, the activator of nitric oxide synthase 1310 comprises a kinase activator 1317. For example, the activator of nitric oxide synthase can include an activator or agonist of a kinase responsible for phosphorylating nitric oxide synthase. For example, the activator of nitric oxide synthase can include an agonist or antagonist of at least one of Akt/protein kinase B (Akt), protein kinase A (PKA), adenosine monophosphate-activated protein kinase (AMPK), protein kinase G, CaMKII, and protein kinase C, kinases known to phosphorylate nitric oxide synthase. See, e.g., Heiss & Dirsch (2014) “Regulation of eNOS enzyme activity by posttranslational modification,” Curr. Pharm. Des. 20:3503-3513, which is incorporated by reference herein.

In an aspect, the activator of nitric oxide synthase 1310 comprises a modulator of posttranslational modification of nitric oxide synthase 1318. For example, the modulator of posttranslational modification of nitric oxide synthase can include an agonist or an antagonist of posttranslational modification of nitric oxide synthase. In an aspect, the activator of nitric oxide synthase comprises a modulator of at least one of acylation, nitrosylation, phosphorylation, acetylation, or glutathionylation of nitric oxide synthase 1319. For example, the activator of nitric oxide synthase can include an agonist or antagonist of a phosphatase or kinase responsible for modulating the phosphorylation state of nitric oxide synthase, as previously described above herein. For example, the activator of nitric oxide synthase can include an activator of acylation of nitric oxide synthase as myristolylation and palmitoylation are required to target nitric oxide synthase to caveolae for optimal activity. See, e.g., Shaul et al. (1996) “Acylation targets endothelial nitric oxide synthase to plasmalemmal caveolae,” J. Biol. Chem., 271:6518-6522, which is incorporated herein by reference. For example, the activator of nitric oxide synthase can include an inhibitor of S-nitrosylation of nitric oxide synthase as the addition of NO to nitric oxide synthase has a negative feedback effect on the activity of nitric oxide synthase. See, e.g., Ravi et al. (2004) “S-nitrosylation of endothelial nitric oxide synthase is associated with monomerization and decreased enzyme activity,” Proc. Natl. Acad. Sci. USA, 101:2619-2624, which is incorporated herein by reference. For example, the activator of nitric oxide synthase can include an activator of acetylation of nitric oxide synthase by calreticulin transacetylase, as acetylation of nitric oxide synthase increases nitric oxide synthase activity. See, e.g., Ponnan et al. (2014) “Comparison of protein acetyltransferase action of CRTAase with the prototypes of HAT,” ScientificWorldJournal February 4; 2014:578956. doi: 10.1155/2014/578956, which is incorporated herein by reference. For example, the activator of nitric oxide synthase can include an inhibitor of S-glutathionylation of nitric oxide synthase, as S-glutathionylation of nitric oxide synthase attenuates the nitric oxide producing capability of nitric oxide synthase. See, e.g., Chen et al. (2010) “S-glutathionylation uncouples eNOS and regulates its cellular and vascular function,” Nature 468:1115-1118, which is incorporated herein by reference.

Bone-targeting complex 1300 includes bone-targeting agent 1320 associated with the activator of nitric oxide synthase 1310. The bone-targeting agent 1320 is configured to or has the properties of selectively accumulating in bone tissue and cells. In an aspect, the bone-targeting agent 1320 includes an osteotropic agent. In an aspect, the bone-targeting agent 1320 includes a “targetor” moiety able to recognize bones cells or components thereof. In an aspect, the bone-targeting agent 1320 comprises bisphosphonate 1321. In an aspect, the bone-targeting agent 1320 includes a derivative of bisphosphonate. In an aspect, the bone-targeting agent 1320 comprises at least one of phosphonate, phosphonic acid, aminomethylphosphonic acid, phosphate, or polyphosphate, as shown in block 1322. In an aspect, the bone-targeting agent 1320 comprises bone-morphogenetic protein (BMP). For example, the bone-targeting agent can include BMP2, BMP4, or BMP7. In an aspect, the bone-targeting agent 1320 comprises hydroxyapatite-binding polypeptide 1323. Non-limiting examples of bone-targeting agents have been described above herein.

In an embodiment, the activator of nitric oxide synthase 1310 is non-covalently associated with the bone-targeting agent 1320. For example, the activator of nitric oxide synthase and the bone-targeting agent can be respectively coupled to one another through non-covalent bonding, e.g., through ionic, hydrogen, or halogen bonding and/or Van der Waals forces, π effects, or hydrophobic interactions. In an embodiment, the activator of nitric oxide synthase 1310 is covalently associated with the bone-targeting agent 1320. For example, the activator of nitric oxide synthase and the bone-targeting agent can be coupled to one another through a covalent or chemical bond.

In some embodiments, the composition includes a linker coupling the activator of nitric oxide synthase 1310 to the bone-targeting agent 1320. In an aspect, the linker includes a first end coupled to the activator of nitric oxide synthase 1310 and a second end coupled to the bone-targeting agent 1320. In an aspect, the linker includes a first end conjugated to the activator of nitric oxide synthase 1310 and a second end conjugated to the bone-targeting agent 1320. In an aspect, the linker comprises a disulfide linker, a carbamate linker, an amide linker, an ester linker, or an ether linker. In an aspect, the linker includes a chemical crosslinker. In an aspect, the linker comprises a cleavable linker. In an aspect, the cleavable linker comprises a stimulus-responsive cleavable linker. In an aspect, the stimulus-responsive cleavable linker comprises at least one of an energy-responsive cleavable linker, a chemically-responsive cleavable linker, or an enzymatically-responsive cleavable linker. Non-limiting aspects of linkers and cleavable linkers have been described above herein.

In an embodiment, the composition including bone-targeting complex 1300 further at least one carrier or excipient mixed with the bone-targeting complex to form at least one of a topical dosage form, an enteral dosage form, or a parenteral dosage form for delivery to a subject. Non-limiting aspects of carrier, excipients, and formulations have been described above herein.

In some embodiments, a bone-targeting complex includes at least a portion of a nitric oxide synthase, a bone-targeting agent, and a linker coupling the at least a portion of the nitric oxide synthase to the bone-targeting agent. In an aspect, the bone-targeting complex is configured to deliver at least a portion of a nitric oxide synthase to bone, e.g., to a bone cell.

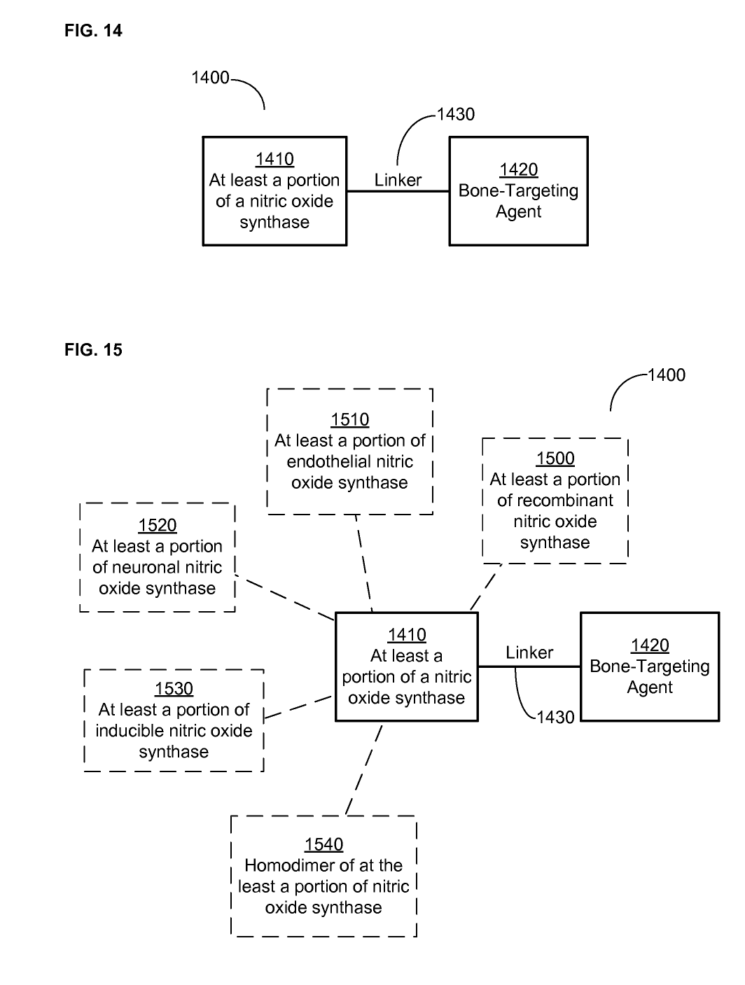

FIG. 14 illustrates aspects of a composition including a bone-targeting complex with at least a portion of a nitric oxide synthase, a bone-targeting agent, and a linker. FIG. 14 shows a block diagram of bone-targeting complex 1400 including at least a portion of a nitric oxide synthase 1410, bone-targeting agent 1420, and linker 1430 coupling the least a portion of a nitric oxide synthase 1410 to the bone-targeting agent 1420.

In an aspect, a bone-targeting complex 1400 includes at least a portion of a nitric oxide synthase 1410. In an aspect, the at least a portion of the nitric oxide synthase includes a full-length version of nitric oxide synthase. The full-length amino acid sequence of endothelial, neuronal, and inducible nitric oxide synthases from various species are available in the National Institutes of Health (NIH) genetic sequence database GenBank® (Benson, et al., “GenBank” Nucleic Acids Research, 2013 January; 41(D1):D36-42, which is incorporated herein by reference). In an aspect, the at least a portion of the nitric oxide synthase includes a truncated version of nitric oxide synthase. In an aspect, the at least a portion of the nitric oxide synthase 1410 is at least a portion of a human nitric oxide synthase. In an aspect, the at least a portion of a nitric oxide synthase 1410 is at least a portion of a mammalian nitric oxide synthase. For example, the at least a portion of the nitric oxide synthase can be derived from a human, simian, a feline, a canine, a bovine, an ovine, a porcine, equine, or other mammalian species. In an aspect, the at least a portion of a nitric oxide synthase 1410 is at least a portion of vertebrate nitric oxide synthase.

In an aspect, the at least a portion of the nitric oxide synthase is derived using standard protein purification methods for purifying a protein from a cell or tissue homogenate. See, e.g., Bredt & Snyder (1990) “Isolation of nitric oxide synthetase, a calmodulin-requiring enzyme,” Proc. Natl. Acad. Sci. USA 87:682-685; Pollock et al. (1991) “Purification and characterization of particulate endothelium-derived relaxing factor synthase form cultured and native bovine aortic endothelial cells,” Proc. Natl. Acad. Sci. USA 88:10480-10484, which are incorporated herein by reference.

FIG. 15 is a block diagram showing further aspects of a bone-targeting complex such as shown in FIG. 14. In an aspect, the at least a portion of the nitric oxide synthase 1410 comprises at least a portion of recombinant nitric oxide synthase 1500. In an aspect, the at least a portion of the nitric oxide synthase is derived using standard recombinant DNA and expression methods combined with standard protein purification methods. See, e.g., Forstermann et al. (1994) “Nitric Oxide Synthase Isozymes: Characterization, purification, molecular cloning, and functions,” Hypertension 23:1121-1131; and Nakane et al. (1995) “Functional expression of three isoforms of human nitric oxide synthase in baculovirus-infected insect cells,” Biochem. Biophys. Res. Comm., 206:511-517, which are incorporated herein by reference.

In an aspect, the at least a portion of a nitric oxide synthase 1410 comprises at least a portion of endothelial nitric oxide synthase 1510. In an embodiment, the at least a portion of endothelial nitric oxide synthase is derived using standard protein purification methods for purifying a protein from a cell or tissue homogenate. In an embodiment, the at least a portion of endothelial nitric oxide synthase includes at least a portion of recombinant endothelial nitric oxide synthase derived using standard recombinant DNA and expression methods combined with standard protein purification methods. See, e.g., Leber et al. (1999) Characterization of recombinant human endothelial nitric-oxide synthase purified from the yeast Pichia pastoris,” J. Biol. Chem. 274:37658-37664, which is incorporated herein by reference.

In an aspect, the at least a portion of a nitric oxide synthase 1410 comprises at least a portion of neuronal nitric oxide synthase 1520. In an embodiment, the at least a portion of neuronal nitric oxide synthase is derived using standard protein purification methods for purifying a protein from a cell or tissue homogenate. In an aspect, the at least a portion of neuronal nitric oxide synthase includes at least a portion of recombinant neuronal nitric oxide synthase derived using standard recombinant DNA and expression methods combined with standard protein purification methods. See, e.g., Charles et al. (1993) “Cloning and expression of a rat neuronal nitric oxide synthase coding sequence in a baculovirus/insect cell system,” Biochm. Biophys. Res. Comm., 196:1481-1489, which is incorporated herein by reference.

In an aspect, the at least a portion of a nitric oxide synthase 1410 comprises at least a portion of inducible nitric oxide synthase 1530. In an embodiment, the at least a portion of inducible nitric oxide synthase is derived using standard protein purification methods for purifying a protein from a cell or tissue homogenate. In an aspect, the at least a portion of inducible nitric oxide synthase includes at least a portion of recombinant inducible nitric oxide synthase derived using standard recombinant DNA and expression methods combined with standard protein purification methods. See, e.g., Lyons et al. (1992) “Molecular cloning and functional expression of an inducible nitric oxide synthase from a murine macrophage cell line,” J. Biol. Chem. 267:6370-6374, which is incorporated herein by reference.

In an aspect, the at least a portion of the nitric oxide synthase is obtained from a commercial (from, e.g., Cayman Chemicals, Ann Arbor, Mich.; Sigma-Aldrich, St. Louis, Mo.; or OriGene, Rockville, Md.). In some instances, the at least a portion of the nitric oxide synthase is obtained from a commercial source as a purified enzyme. In some instances, the recombinant nitric oxide synthase is obtained from a commercial source as a recombinant DNA construct in a bacterial, (e.g., E. coli), yeast, or Baculovirus expression system.

In an aspect, the at least a portion of a nitric oxide synthase 1410 includes at least a portion of an NO-forming portion of nitric oxide synthase. In an aspect, the at least a portion of the nitric oxide synthase 1410 includes a homodimer of at least a portion of nitric oxide synthase 1540. See, e.g., Baek et al. (1993) “Macrophage nitric oxide synthase subunits: purification, characterization, and role of prosthetic groups and substrate in regulating their association into a dimeric enzyme,” J. Biol. Chem. 268:21120-21129, which is incorporated herein by reference.

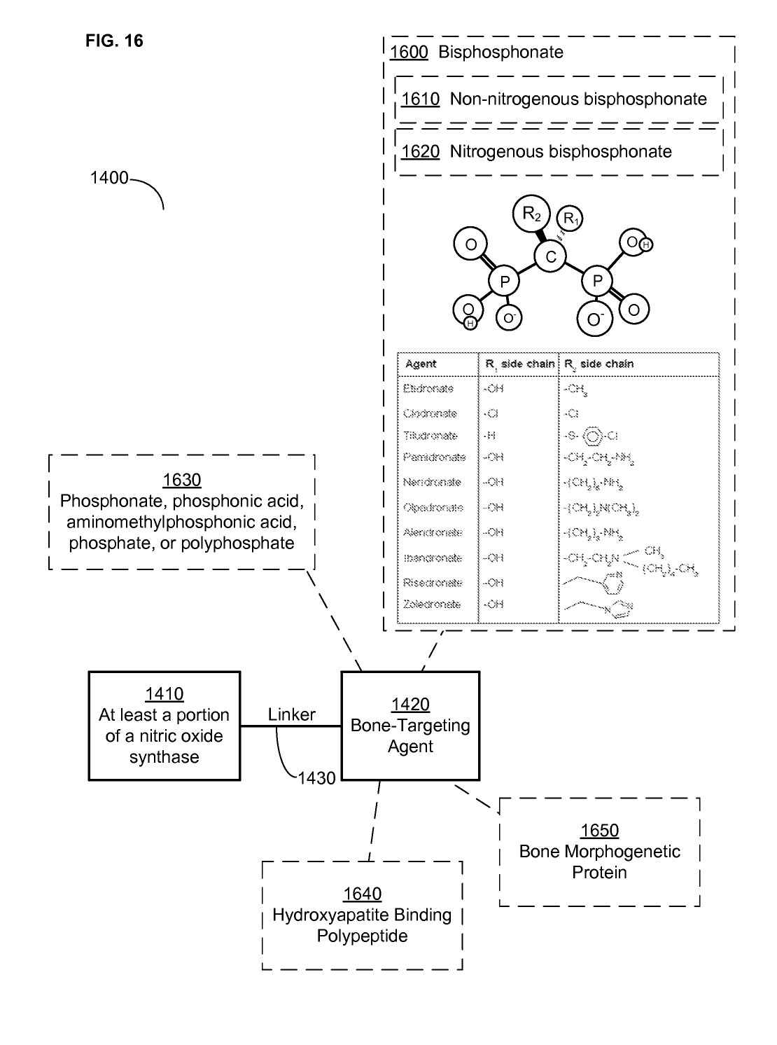

FIG. 16 illustrates further aspects of bone-targeting complex 1400. FIG. 16 shows a block diagram of bone-targeting complex 1400 including at least a portion of a nitric oxide synthase 1410, bone-targeting agent 1420, and linker 1430. Also shown are non-limiting embodiments of bone-targeting agents.

Bone-targeting complex 1400 includes bone-targeting agent 1420. The bone-targeting agent is configured to or has the properties of selectively accumulating in bone tissue and cells. In an aspect, the bone-targeting agent includes an osteotropic agent. In an aspect, the bone-targeting agent includes a “targetor” moiety able to recognize bones cells or components thereof.

In an aspect, the bone-targeting agent 1420 comprises bisphosphonate 1600. In an aspect, the bone-targeting agent 1420 includes a bisphosphonate derivative. In an aspect, bone-targeting agent 1420 comprises a non-nitrogenous bisphosphonate 1610. Non-limiting examples of non-nitrogenous or non-nitrogen-containing bisphosphonates include etidronate, clodronate, and tiludronate. In an aspect, bone-targeting agent 1420 comprises a nitrogenous bisphosphonate 1620. Non-limiting examples of nitrogenous bisphosphonates include pamidronate, neridronate, olpadronate, alendronate, ibandronate, risedronate, and zoledronate. Other non-limiting aspects of bisphosphonates have been described above herein.

In an aspect, the bone-targeting agent 1420 includes an organic phosphate. In an aspect, the bone-targeting agent 1420 comprises phosphonate, phosphonic acid, aminomethylphosphonic acid, phosphate, or polyphosphate, as shown in block 1630. In an aspect, the bone-targeting agent includes sodium orthophosphate or hydroxyethylidene diphosphonate. In an aspect, the bone-targeting agent includes a phosphate derivative. For example, the bone-targeting agent can include at least one of carbamyl phosphate, acetyl phosphate, propionyl phosphate, and butyryl phosphate, phosphono-acetic acid.

In an aspect, the bone-targeting agent 1420 includes calcium. In an aspect, the bone-targeting agent includes members of the IIA family of the periodic table which carry the same divalent charge as elemental calcium and are incorporated into bone matrix directly. For example, the bone-targeting agent can include strontium. For example, the bone-targeting agent can include radium.

In an aspect, the bone-targeting agent 1420 comprises a hydroxyapatite-binding polypeptide 1640. In an aspect, the bone-targeting agent includes negatively charged calcium-binding domains. For example, a hydroxyapatite-binding polypeptide can include a plurality of aspartic acid moieties (polyaspartate). In an aspect, a hydroxyapatite-binding polypeptide includes a plurality of glutamic acids (polyglutamate). For example, a string of aspartic acids (poly(aspartic acid)) can be conjugated to an inhibitor of nitric oxide synthase uncoupling to confer bone-targeting, bone-seeking, or osteotrophic properties to the complex. Other non-limiting examples of hydroxyapatite-binding polypeptides are described in U.S. Pat. No. 8,022,040 to Bertozzi et al. titled “Hydroxyapatite-binding peptides for bone growth and inhibition,” which is incorporated herein by reference.

In an aspect, the bone-targeting agent 1420 comprises a bone morphogenetic protein 1650. For example, the bone-targeting agent can include any of a number of bone morphogenetic proteins known to interact with receptors on bone and/or cartilage or associated precursor cells to induce formation of bone and/or cartilage. In an aspect, the bone morphogenetic protein includes BMP2 or BMP4. In an aspect, the bone morphogenetic protein includes BMP7. In an aspect, the bone-targeting agent includes a recombinant form of a bone morphogenetic protein. For example, the bone-targeting agent can include recombinant human BMP2 (rhBMP2) or recombinant human BMP7 (rhBMP7). Non-limiting examples of bone morphogenetic proteins include BMP1, BMP2, BMP3, BMP4, BMP5, BMP6, BMP7, BMP8a, BMP8b, BMP10, and BMP15. See, e.g., Ducy & Karsenty (2000) “The family of bone morphogenetic proteins,” Kidney International 57:2207-2214; Granjeiro et al. (2005) “Bone morphogenetic proteins: from structure to clinical use,” Braz. J. Med. Biol. Res. 38:1463-1473, which are incorporated herein by reference.

In an aspect, a bone morphogenetic protein is fused with at least a portion of a nitric oxide synthase using standard recombinant DNA techniques to form a fusion protein with bone-targeting properties and nitric oxide synthase activity. In an aspect, a bone-morphogenetic protein is linked to at least a portion of a nitric oxide synthase through a chemical crosslinking reagent.

Bone-targeting complex 1400 includes linker 1430. In an aspect, linker 1430 includes a peptidyl linker of two or more amino acids. In an aspect, linker 1430 includes an oligonucleotide or oligomer of two or more nucleotides. In an aspect, linker 1430 includes a ligand/receptor pair. In an aspect, linker 1430 includes an oligosaccharide. In an aspect, linker 1430 includes an acyl chain. In general, the linker is configured to couple the at least a portion of the nitric oxide synthase to the bone-targeting agent.

In an embodiment, the at least a portion of a nitric oxide synthase 1410 is coupled to a first end of the linker 1430 and the bone-targeting agent 1420 is coupled to a second end of the linker 1430. For example, the at least a portion of the nitric oxide synthase and the bone-targeting agent can be respectively coupled to the first and the second end of the linker through non-covalent bonding, e.g., through ionic, hydrogen, or halogen bonding and/or Van der Waals forces, π effects, or hydrophobic interactions. For example, the at least a portion of the nitric oxide synthase and the bone-targeting agent can be respectively coupled to the first and the second end of the linker through a covalent or chemical bond. In an embodiment, the at least a portion of the nitric oxide synthase 1410 is conjugated to a first end of the linker 1430 and the bone-targeting agent 1420 is conjugated to a second end of the linker 1430.

In an aspect, linker 1430 is configured to link the at least a portion of the nitric oxide synthase 1410 to a bone-targeting agent 1420. In an aspect, linker 1430 comprises a disulfide linker, a carbamate linker, an amide linker, an ester linker, or an ether linker. In an aspect, linker 1430 includes a chemical crosslinker. For example, the at least a portion of the nitric oxide synthase can be linked to a bone morphogenetic peptide using a chemical crosslinking reagent. A number of examples of chemical crosslinking reagents are commercially available from, e.g., Thermo Fisher Scientific, Waltham, Mass. Also see, e.g., “Thermo Scientific Pierce Crosslinking Technical Handbook” published by Thermo Fisher Scientific and incorporated herein by reference.

In an embodiment, linker 1430 includes a ligand/receptor pair. For example, the linker can include a biotin/avidin pair, wherein the avidin is covalently attached to the at least a portion of the nitric oxide synthase and the biotin is covalently attached to the bone-targeting agent. Ligand/receptor pairs can include antigen/antibody, co-factor/protein, and substrate/enzyme pairs. Non-limiting examples include biotin/avidin, biotin/streptavidin, FK506/FK506-binding protein (FKBP), rapamycin/FKBP, cyclophilin/cyclosporine, and glutathione/glutathione transferase pairs.

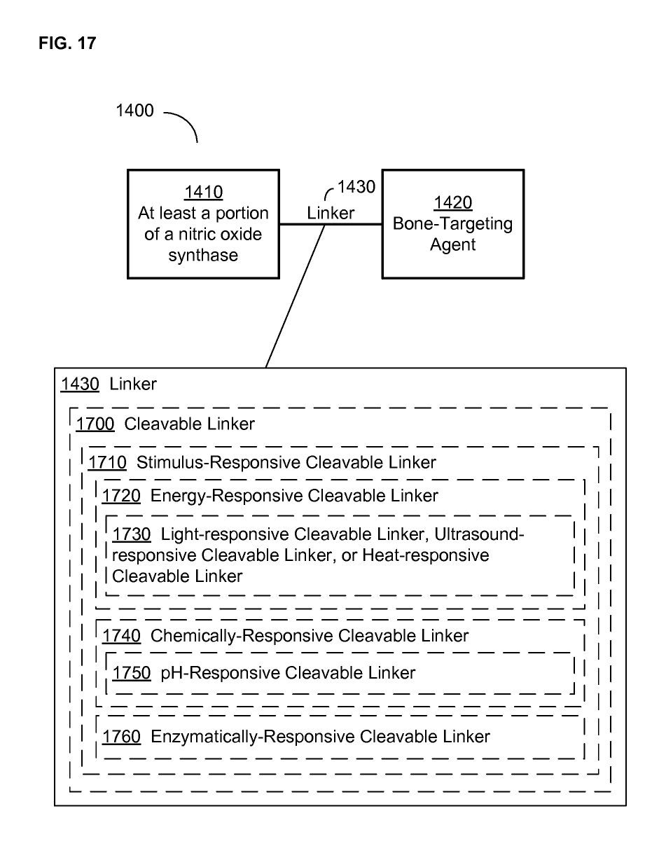

FIG. 17 illustrates further aspects of bone-targeting complex 1400. FIG. 17 shows a block diagram of bone-targeting complex 1400 including at least a portion of a nitric oxide synthase 1410, a bone-targeting agent 1420, and linker 1430. Also shown are non-limiting embodiments of linker 1430.

In an aspect, linker 1430 comprises cleavable linker 1700. For example, the linker can include a cleavable linker that is cleaved at some point after administration of the composition to a subject to release the at least a portion of the nitric oxide synthase from the bone-targeting agent. In an aspect, the cleavable linker is cleavable under extracellular conditions, releasing the at least a portion of the nitric oxide synthase from the bone-targeting agent in an extracellular environment. In an aspect, the cleavable linker is cleavable under intracellular conditions, releasing the at least a portion of the nitric oxide synthase from the bone-targeting agent in an intracellular environment. For example, the cleavable linker can be a peptidyl linker that is cleaved enzymatically by an intracellular peptidase or protease. For example, the cleavable linker can be cleaved in response to a pH change associated with an organelle, e.g., the lysosome, endosome, peroxisome, or caveolea.

In an aspect, cleavable linker 1700 comprises a stimulus-responsive cleavable linker 1710. For example, the cleavable linker can be responsive to an endogenous stimulus, e.g., a stimulus emanating from the subject to whom the composition has been administered. Non-limiting examples of stimuli emanating from the subject include pH changes, temperature changes, and enzymatic or other chemical activity. For example, the cleavable linker can be responsive to an exogenous stimulus, e.g., a stimulus emanating from outside the subject to whom the composition has been administered. Non-limiting examples of stimuli emanating from outside the subject include energy stimuli, e.g., light, ultrasound, or heat.

In an aspect, stimulus-responsive cleavable linker 1710 comprises an energy-responsive cleavable linker 1720. For example, the cleavable linker can be responsive to an energy stimulus. Non-limiting examples of energy stimuli include electromagnetic energy, acoustic energy, magnetic energy, light energy, radiofrequency energy, and/or microwave energy. In an aspect, the energy-responsive cleavable linker 1720 comprises at least one of a light-responsive cleavable linker, an ultrasound-responsive cleavable linker, or heat-responsive cleavable linker 1730. In an aspect, the energy-responsive cleavable linker 1720 includes a light-responsive cleavable linker. In an aspect, the energy-responsive cleavable linker 1720 includes an ultrasound-responsive cleavable linker. In an aspect, the energy-responsive cleavable linker 1720 includes a heat-responsive cleavable linker. Non-limiting examples of light-responsive, ultrasound-responsive, and heat-responsive cleavable linkers have been described above herein.

In an aspect, stimulus-responsive cleavable linker 1720 comprises a chemically-responsive cleavable linker 1740. For example, the stimulus-responsive cleavable linker can be responsive to a chemical reaction or condition. For example, the chemically-responsive cleavable linker can be configured to be responsive to oxidizing conditions, reducing conditions, and/or pH conditions. In an aspect, the chemically-responsive cleavable linker 1740 comprises a pH-responsive cleavable linker 1750. For example, the cleavable linker can include a pH-sensitive cleavable linker, e.g., sensitive to hydrolysis/cleavage at certain pH values. For example, the pH-responsive cleavable linker can be responsive to changes in pH as the composition is brought into a cell or into a subcellular organelle, e.g., the lysosome. For example, the cleavable linker can include an acid-labile linker responsive to an acidic pH (e.g., an amino-sulfhydryl, thioether, hydrazone, semicarbazone, thiosemicarbazone, cis-aconitic amide, orthoester, acetal, ketal, or the like). In an aspect, the chemically-responsive cleavable linker 1740 is responsive to oxidation. For example, the chemically-responsive cleavable linker can be cleavable in response to the presence of superoxide. In an aspect, the chemically-responsive cleavable linker 1740 can include a disulfide group and be responsive to reducing conditions. Non-limiting examples of chemically-responsive cleavable linkers have been described above herein.

In an aspect, the stimulus-responsive cleavable linker 1710 comprises an enzymatically-responsive cleavable linker 1760. For example, the cleavable linker can be responsive to an enzymatic activity endogenous to the subject to whom the composition has been administered. For example, the cleavable linker can be responsive to an enzymatic activity specific to a target cell, e.g., a bone cell, to which the composition is directed or accumulates. For example, the enzymatically-responsive cleavable linker can include a peptidase- or protease-sensitive dipeptide or oligopeptide sensitive to cleavage by a peptidase or protease enzyme. In an aspect, the enzymatic stimulus, e.g., a peptidase or protease enzyme, is present in the intracellular environment, e.g., within a lysosome, endosome, or caveolea. In an aspect, the enzymatically-responsive cleavable linker is designed such that there is little or no cleavage of the linker in the plasma. In an aspect, the enzymatically-responsive cleavable linker is cleavable in response to a bone-specific peptidase or protease. For example, the enzymatically-responsive cleavable linker can include a peptide sequence cleavable by cathepsin K, a cysteine protease. For example, the enzymatically-responsive cleavable linker can include a peptide sequence cleavable by one or more matrix metalloproteinases. Non-limiting aspects of enzymatically-responsive cleavable linkers have been described above herein.

In some embodiments, a bone-targeting complex includes a cell-penetrating means. See, e.g., Torchilin (2008) “Intracellular delivery of protein and peptide therapeutics,” Drug Discovery Today: Technologies, 5: e95-e013, which is incorporated herein by reference. For example, the bone-targeting complex can include a cell-penetrating means that facilitates entry of the complex into a cell, e.g., a bone cell. In an embodiment, a bone-targeting complex including an inhibitor of nitric oxide synthase uncoupling, a bone-targeting agent, and a linker further includes a cell-penetrating means. In an embodiment, a bone-targeting complex including an activator of nitric oxide synthase, a bone-targeting agent, and a cleavable linker further includes a cell-penetrating means. In an embodiment, a bone-targeting complex including at least a portion of nitric oxide synthase, a bone-targeting agent, and a linker further includes a cell-penetrating means.

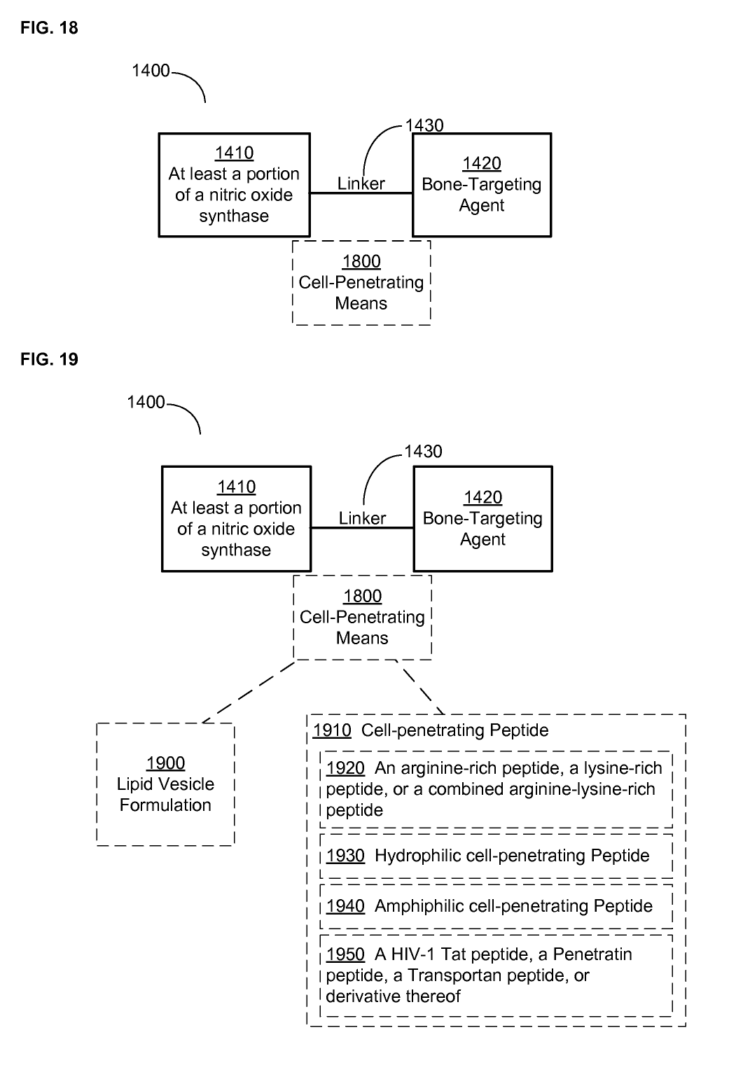

FIG. 18 illustrates further aspects of bone-targeting complex 1400. FIG. 18 shows a block diagram of bone-targeting complex 1400 including at least a portion of a nitric oxide synthase 1410, a bone-targeting agent 1420, and linker 1430 coupling the at least a portion of the nitric oxide synthase 1410 to the bone-targeting agent 1420. In an aspect, bone-targeting complex 1400 further includes cell-penetrating means 1800 associated with the bone-targeting complex 1400. In an aspect, the cell-penetrating means 1800 is associated with the at least a portion of the nitric oxide synthase 1410 and/or bone-targeting agent 1420. For example, the cell-penetrating means can be coupled to or conjugated to the at least a portion of the nitric oxide synthase and/or to the bone-targeting agent. A cell-penetrating means that includes a lipid vesicle may at least partially encapsulate the bone-targeting complex. A cell-penetrating means that includes a cell-penetrating peptide can be incorporated into the amino acid sequence of the at least a portion of the nitric oxide synthase. In general, the cell-penetrating means is configured to facilitate passage of the bone-targeting complex across a cellular membrane and into a target cell.

FIG. 19 illustrates further aspects of bone-targeting complex 1400 including a cell-penetrating means 1800. FIG. 19 shows a block diagram of bone-targeting complex 1400 including at least a portion of a nitric oxide synthase 1410, a bone-targeting agent 1420, linker 1430, and cell-penetrating means 1800. Also shown are non-limiting embodiments of cell-penetrating means.

In an aspect, the cell-penetrating means 1800 comprises a lipid vesicle formulation 1900. For example, the bone-targeting complex can be formulated with lipid vesicles to facilitate entry of the complex into a bone cell. In an aspect, the lipid vesicle formulation 1900 includes liposomes. For example, the bone-targeting complex can be formulated in liposomes formed from phospholipids, e.g., phosphatidylserine or phosphatidylinositol. In an aspect, the lipid vesicle includes at least one of liposomes, solid lipid nanoparticle, lipid microbubbles, inverse lipid micelles, cochlear liposomes, lipid microtubules, or lipid microcylinders. In an aspect, the lipid vesicles include at least one of small unilamellar vesicles, large unilamellar vesicles, or multilamellar vesicles. See, e.g., Pisal et al. (2010) “Delivery of Therapeutic Proteins,” J. Pharm. Sci. 99:2557-2575, which is incorporated herein by reference.

In an aspect, the lipid vesicle formulation 1900 includes archeosomes comprised of polar lipids of archaebacterial. For example, the bone-targeting complex can be formulated in archeosomes including archae lipids, archaeol (diether) lipids, and/or caldarchaeol (tetraether) lipids. In an aspect, the lipid vesicle formulation 1900 includes cochelates. For example, the bone-targeting complex can be formulated in cochelates including a cylindrical lipid bilayer of negatively charges lipids, e.g., phosphatidylserine, stabilized with inorganic multivalent cations, e.g., zinc and calcium, and other organic multivalent cations. In an aspect, the lipid vesicle formulation 1900 includes cubosomes. For example, the bone-targeting complex can be formulated in cubosomes including self-assembled cubic crystals of detergents. In an aspect, the lipid vesicle formulation 1900 includes ethosomes. For example, the bone-targeting complex can be formulated in ethosomes including a hydroalcoholic core of ethanol. In an aspect, the lipid vesicle formulation 1900 includes exosomes. For example, the bone-targeting complex can be formulated in exosomes including phospholipid vesicles released by normal or tumor cells. In an aspect, the lipid vesicle formulation 1900 includes immunoliposomes. For example, the bone-targeting complex can be formulated in immunoliposomes including an antibody or antibody fragment associated with the liposomes that targets the liposome to a tissue or cell type, e.g., bone. In an aspect, the lipid vesicle formulation 1900 includes transferosomes. For example, the bone-targeting complex can be formulated in transferosomes including phosphatidylcholine and surfactants. For a review of various liposome technologies, see, e.g., Madni et al. (2014) “Liposomal Drug Delivery: A Versatile Platform for Challenging Clinical Applications,” J. Pharm. Pharm. Sci. 17:401-426, which are incorporated herein by reference.

In an aspect, the cell-penetrating means 1800 comprises a cell-penetrating peptide 1910. In an aspect, the cell-penetrating peptide 1910 includes a protein transduction domain. In an aspect, the cell-penetrating peptide 1910 includes a “Trojan” peptide. In an aspect, the cell-penetrating peptide 1910 includes a membrane translocation sequence. For example, the bone-targeting complex can include a cell-penetrating peptide that facilitates entry of the complex into a cell (e.g., a bone cell). See, e.g., Bechara & Sagan (2013) “Cell-penetrating peptides: 20 years later, where do we stand?,” FEBS Letters, 587:1693-1702, which is incorporated herein by reference.

In an aspect, the cell-penetrating peptide is associated with the at least a portion of the nitric oxide synthase and/or the bone-targeting agent. In an aspect, the cell-penetrating peptide associated with the bone-targeting complex in a non-covalent manner. See, e.g., Keller et al. (2013) “Relationships between cargo, cell penetrating peptides and cell types for uptake of non-covalent complexes into live cells,” Pharmaceuticals 6:184-203, which is incorporated herein by reference. In an aspect, the cell-penetrating peptide is cross-linked to the at least a portion of the nitric oxide synthase and/or the bone-targeting agent through a standard crosslinking reagent. In an aspect, the cell-penetrating peptide is expressed as part of a recombinant fusion protein with the at least a portion of the nitric oxide synthase and/or the bone-targeting agent.

In an aspect, the cell-penetrating peptide comprises an arginine-rich peptide, a lysine-rich peptide, or a combined arginine-lysine-rich peptide 1920. In an aspect, the cell-penetrating peptide comprises a hydrophilic cell-penetrating peptide 1930.

For example, the hydrophilic cell-penetrating peptide can be mainly composed of hydrophilic amino acids, e.g., arginine and lysine amino acids. Non-limiting examples of hydrophilic cell-penetrating peptides include penetratin, antennapedia PTD (protein transduction domain), HIV-1 Tat peptide, SynB1, SynB3, PTD-4, PTD-5, FHV (flock house virus) coat peptide, BMV (Brome mosaic virus) Gag peptide, HTLV-II (human T-lymphotropic virus II) Rex, D-tat, or R9-Tat. In an aspect, the cell-penetrating peptide comprises an amphiphilic cell-penetrating peptide 1940. For example, the amphiphilic cell-penetrating peptide can be rich in lysine residues. Non-limiting examples of amphiphilic cell-penetrating peptides includes transportan, MAP (model amphipathic peptide), SBP (single-based peptide), FBP (fusion sequence based-peptide), MPG, Pep-1, Pep-2. Other non-limiting examples of cell-penetrating peptides includes BAC715-24, Buforin II, CADY, CCMV Gag, Cell Penetrating ARF peptide, D-TAT, HIV-1 Rev, HN-1, K-FGF, Ku70, P22 N, Pen2W2F, pls1-1, pVEC, SAP, and VP22. In an aspect, the cell-penetrating peptide comprises a HIV-1 Tat (trans-activator of transcription) peptide, a penetratin peptide, a transportan peptide, or derivatives thereof 1950. See, e.g., Ciobanasu et al. (2010) “Cell-penetrating HIV1 TAT peptides can generate pores in model membranes,” Biophysical J. 99:153-162, which is incorporated herein by reference.

In an aspect, the cell-penetrating peptide includes periodic sequences. For example, the cell-penetrating peptide can include an amino acid sequence motif that is replicated several times, e.g., pVec and pep-1. Non-limiting examples of periodic sequences include polyarginines R×N (4<N<17), polylysines K×N (4<N<17), (RAca)6R, (RAbu)6R, (RG)6R, (RM)6R, (RT)6R, (RS)6R, R10, (RA)6R, or R7. In an aspect, the cell-penetrating peptide is a known cell-penetrating peptide, non-limiting examples of which have been described above. Additional examples of cell-penetrating peptides can be found at the cell-penetrating peptide website “CPPsite” with a URL of http://crdd.osdd.net/raghava/cppsite/ and referenced in Gautam et al. (2012) “CPPsite: A curated database of cell penetrating peptides,” Database, 2012 Mar. 7; 2012:bas015. doi: 10.1093/database/bas015, which is incorporated herein by reference.

In an aspect, the cell-penetrating peptide includes any of a number of cell-penetrating peptides available from commercial sources (from, e.g., Creative Peptide, Shirley, N.Y.; Phoenix Pharmaceuticals, Inc., Burlingame, Calif.).

In an aspect, the cell-penetrating peptide is generated de novo. For example, the cell-penetrating peptide can be designed based on homology to known cell-penetrating peptides, e.g., peptides composed primarily of lysine and/or arginine. See, e.g., Sanders et al. (2011) “Prediction of cell penetrating peptides by support vector machines,” PLoS Comput. Biol. 7(7) e1002101, which is incorporated herein by reference. For example, the cell-penetrating peptide can be derived from a phage display library. See, e.g., Shi et al. (2014) “A survey on ‘Trojan Horse’ peptides: Opportunities, issues and controlled entry to ‘Troy’,” J. Controlled Release, 194:53-70; Jarver et al. (2012) “Peptide-mediated cell and in vivo delivery of antisense oligonucleotides and siRNA,” Molecular Therapy-Nucleic Acids 1, e27; doi:10.1038/mtna.2012.18, which are incorporated herein by reference.

In an embodiment, the composition including bone-targeting complex 1400 further includes at least one carrier or excipient mixed with the bone-targeting complex to form at least one of a topical dosage form, an enteral dosage form, or a parenteral dosage form for delivery to a subject. Non-limiting aspects of carrier, excipients, and formulations have been described above herein.

In some embodiments, a composition includes a bone-targeting complex including at least a portion of a nitric oxide synthase, a bone-targeting agent, and a cell-penetrating means.

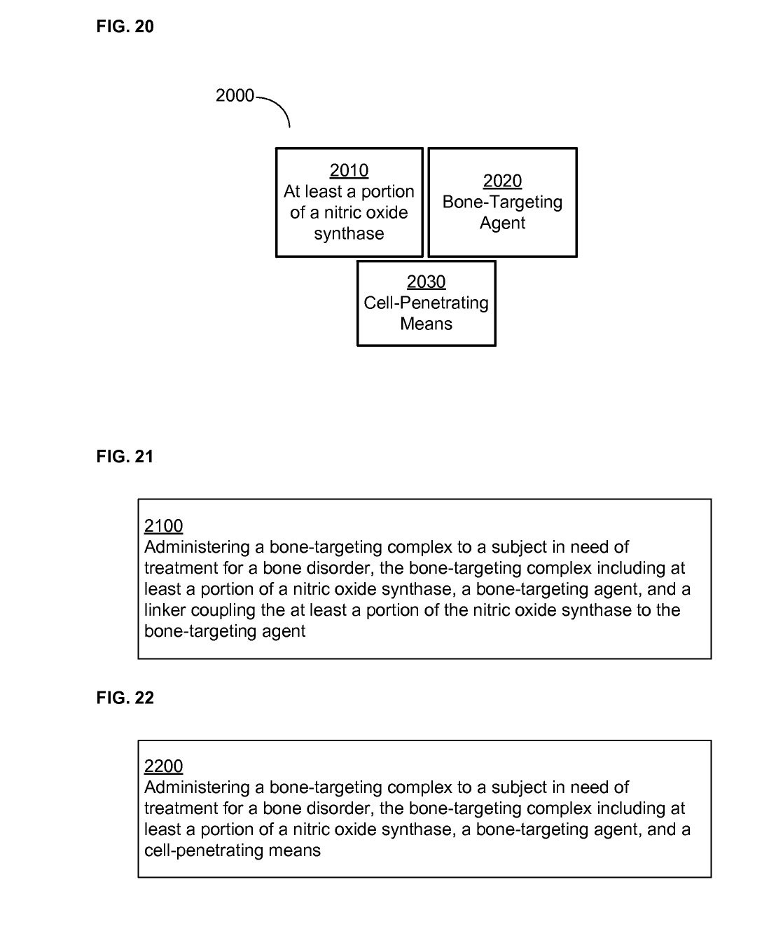

FIG. 20 illustrates aspects of a composition including a bone-targeting complex. FIG. 20 shows a block diagram of bone-targeting complex 2000 including at least a portion of a nitric oxide synthase 2010, a bone-targeting agent 2020, and a cell-penetrating means 2030.

Bone-targeting complex 2000 includes at least a portion of a nitric oxide synthase 2010. In an aspect, the at least a portion of a nitric oxide synthase includes a full-length version of nitric oxide synthase. In an aspect, the at least a portion of a nitric oxide synthase includes a truncated version of nitric oxide synthase. In an aspect, the at least a portion of a nitric oxide synthase 2010 includes at least a portion of endothelial nitric oxide synthase, neuronal nitric oxide synthase, or inducible nitric oxide synthase. In an aspect, the at least a portion of a nitric oxide synthase 2010 is at least a portion of a human nitric oxide synthase. In an aspect, the at least a portion of a nitric oxide synthase 2010 is at least a portion of a mammalian nitric oxide synthase. For example, the at least a portion of the nitric oxide synthase can be derived from a human, simian, a feline, a canine, a bovine, an ovine, a porcine, equine, or other mammalian species. In an aspect, the at least a portion of a nitric oxide synthase 2010 is at least a portion of a vertebrate nitric oxide synthase. The full-length amino acid sequence of endothelial, neuronal, and inducible nitric oxide synthases from various species are available in the National Institutes of Health (NIH) genetic sequence database GenBank® (Benson, et al., “GenBank” Nucleic Acids Research, 2013 January; 41(D1):D36-42, which is incorporated herein by reference).

In an aspect, the at least a portion of a nitric oxide synthase 2010 is derived using standard protein purification methods for purifying a protein from a cell or tissue homogenate. In an aspect, the at least a portion of a nitric oxide synthase 2010 comprises at least a portion of recombinant nitric oxide synthase. In an aspect, the at least a portion of the nitric oxide synthase is derived using standard recombinant DNA and expression methods combined with standard protein purification methods. Non-limiting aspects of deriving at least a portion of endothelial, inducible, and/or neuronal nitric oxide synthase have been described above herein. In an aspect, the at least a portion of a nitric oxide synthase 2010 is obtained from a commercial source (from, e.g., Cayman Chemicals, Ann Arbor, Mich.; Sigma-Aldrich, St. Louis, Mo.; or OriGene, Rockville, Md.). In some instances, the at least a portion of a nitric oxide synthase is obtained from a commercial source as a purified enzyme. In some instances, the recombinant nitric oxide synthase is obtained from a commercial source as a recombinant DNA construct in a bacterial, (e.g., E. coli), yeast, or Baculovirus expression system.

Bone-targeting complex 2000 includes bone-targeting agent 2020. In an aspect, the bone-targeting agent 2020 comprises at least one of bisphosphonate, a hydroxyapatite-binding peptide, or bone-morphogenetic protein. The bone-targeting agent is configured to or has the properties of selectively accumulating in bone tissue and cells. In an aspect, the bone-targeting agent includes an osteotropic agent. In an aspect, the bone-targeting agent includes a “targetor” moiety able to recognize bones cells or components thereof.

In an aspect, the bone-targeting agent 2020 comprises bisphosphonate. In an aspect, the bone-targeting agent 2020 includes a bisphosphonate derivative. In an aspect, the bone-targeting agent 2020 comprises a non-nitrogenous bisphosphonate. In an aspect, the bone-targeting agent 2020 comprises a nitrogenous bisphosphonate. Non-limiting aspects of bisphosphonates have been described above herein.

In an aspect, the bone-targeting agent 2020 includes an organic phosphate. In an aspect, the bone-targeting agent 2020 comprises phosphonate, phosphonic acid, aminomethylphosphonic acid, phosphate, or polyphosphate. In an aspect, the bone-targeting agent includes sodium orthophosphate or hydroxyethylidene diphosphonate. In an aspect, the bone-targeting agent includes a phosphate derivative (e.g., carbamyl phosphate, acetyl phosphate, propionyl phosphate, or butyryl phosphate, phosphono-acetic acid.

In an aspect, the bone-targeting agent 2020 includes calcium. In an aspect, the bone-targeting agent includes members of the IIA family of the periodic table which carry the same divalent charge as elemental calcium and are incorporated into bone matrix directly (e.g., strontium or radium).

In an aspect, the bone-targeting agent 2020 comprises a bone morphogenetic protein (BMP). For example, the bone-targeting agent can include any of a number of bone morphogenetic proteins known to induce formation of bone and/or cartilage. Non-limiting examples of bone morphogenetic proteins include BMP1, BMP2, BMP3, BMP4, BMP5, BMP6, BMP7, BMP8a, BMP8b, BMP10, and BMP15.

In an aspect, the bone-targeting agent 2020 comprises a hydroxyapatite-binding polypeptide. In an aspect, the bone-targeting agent includes negatively charged calcium-binding domains (e.g., a plurality of aspartic acid moieties (polyaspartate)). In an aspect, a hydroxyapatite-binding polypeptide includes a plurality of glutamic acids (polyglutamate). Other non-limiting examples of hydroxyapatite-binding polypeptides are described in U.S. Pat. No. 8,022,040 to Bertozzi et al. titled “Hydroxyapatite-binding peptides for bone growth and inhibition,” which is incorporated herein by reference.

Bone-targeting complex 2000 further includes cell-penetrating means 2030. In an aspect, the cell-penetrating means 2030 is associated with the at least a portion of a nitric oxide synthase 2010 and/or the bone-targeting agent 2020. A cell-penetrating means that includes a lipid vesicle may at least partially encapsulate the bone-targeting complex. A cell-penetrating means that includes a cell-penetrating peptide can be incorporated into the amino acid sequence of the at least a portion of the nitric oxide synthase and/or the bone-targeting agent. In general, the cell-penetrating means is configured to facilitate passage of the bone-targeting complex across a cellular membrane and into a target cell.

In an aspect, the cell-penetrating means 2030 comprises a lipid vesicle formulation. For example, the bone-targeting complex 2000 can be formulated with lipid vesicles to facilitate entry of the complex into a bone cell. In an aspect, the lipid vesicle formulation includes liposomes. For example, the bone-targeting complex 2000 can be formulated in liposomes formed from phospholipids, e.g., phosphatidylserine or phosphatidylinositol. In an aspect, the cell-penetrating means 2030 includes lipid vesicles that are at least one of liposomes, solid lipid nanoparticle, lipid microbubbles, inverse lipid micelles, cochlear liposomes, lipid microtubules, or lipid microcylinders. In an aspect, the cell-penetrating means 2030 includes lipid vesicles that are small unilamellar vesicles, large unilamellar vesicles, or multilamellar vesicles. See, e.g., Pisal et al. (2010) “Delivery of Therapeutic Proteins,” J. Pharm. Sci. 99:2557-2575, which is incorporated herein by reference. In an aspect, the cell-penetrating means 2030 includes lipid vesicles that are at least one of archeosomes, cochelates, cubosomes, ethosomes, exosomes, immunoliposomes, or transferosomes. Non-limiting aspects of lipid vesicles have been described above herein.

In an aspect, the cell-penetrating means 2030 comprises a cell-penetrating peptide. In an aspect, the cell-penetrating means 2030 includes a cell-penetrating peptide that is a protein transduction domain, a “Trojan” peptide, or a membrane translocation sequence. For example, the bone-targeting complex can include a cell-penetrating peptide that facilitates entry of the complex into a cell (e.g., a bone cell). See, e.g., Bechara & Sagan (2013) “Cell-penetrating peptides: 20 years later, where do we stand?,” FEBS Letters, 587:1693-1702, which is incorporated herein by reference.

In an aspect, cell-penetrating means 2030 includes a cell-penetrating peptide associated with the at least a portion of a nitric oxide synthase 2010 and/or the bone-targeting agent 2020. In an aspect, the cell-penetrating peptide associates with the bone-targeting complex in a non-covalent manner. See, e.g., Keller et al. (2013) “Relationships between cargo, cell penetrating peptides and cell types for uptake of non-covalent complexes into live cells,” Pharmaceuticals 6:184-203, which is incorporated herein by reference. In an aspect, the cell-penetrating peptide is expressed as part of a recombinant fusion protein with the at least a portion of the nitric oxide synthase and/or the bone-targeting agent.

In an aspect, cell-penetrating means 2030 includes a cell-penetrating peptide that is an arginine-rich peptide, a lysine-rich peptide, or a combined arginine-lysine-rich peptide. In an aspect, cell-penetrating means 2030 includes a cell-penetrating peptide that is a HIV-1 Tat (trans-activator of transcription) peptide, a penetratin peptide, a transportan peptide, or a derivative thereof. In an aspect, cell-penetrating means 2030 includes a cell-penetrating peptide that is a hydrophilic cell-penetrating peptide. For example, the hydrophilic cell-penetrating peptide can be mainly composed of hydrophilic amino acids, e.g., arginine and lysine amino acids. In an aspect, cell-penetrating means 2030 includes a cell-penetrating peptide that is an amphiphilic cell-penetrating peptide. For example, the amphiphilic cell-penetrating peptide can be rich in lysine residues. Non-limiting examples of hydrophilic and amphiphilic cell-penetrating peptides have been described above herein.

In an aspect, cell-penetrating means 2030 includes a cell-penetrating peptide that includes periodic sequences. For example, the cell-penetrating peptide can include an amino acid sequence motif that is replicated several times, e.g., pVec and pep-1. Non-limiting examples of periodic sequences include polyarginines R×N (4<N<17), polylysines K×N (4<N<17), (RAca)6R, (RAbu)6R, (RG)6R, (RM)6R, (RT)6R, (RS)6R, R10, (RA)6R, or R7. In an aspect, the cell-penetrating peptide is a known cell-penetrating peptide, non-limiting examples of which have been described above. Additional examples of cell-penetrating peptides can be found at the cell-penetrating peptide website “CPPsite” with a URL of http://crdd.osdd.net/raghava/cppsite/ and referenced in Gautam et al. (2012) “CPPsite: A curated database of cell penetrating peptides,” Database, 2012 Mar. 7; 2012:bas015. doi: 10.1093/database/bas015, which is incorporated herein by reference. In an aspect, the cell-penetrating peptide includes any of a number of cell-penetrating peptides available from commercial sources (from, e.g., Creative Peptide, Shirley, N.Y.; Phoenix Pharmaceuticals, Inc., Burlingame, Calif.).

In an aspect, cell-penetrating means 2030 includes a cell-penetrating peptide that is generated de novo. For example, the cell-penetrating peptide can be designed based on homology to known cell-penetrating peptides, e.g., peptides composed primarily of lysine and/or arginine. See, e.g., Sanders et al. (2011) “Prediction of cell penetrating peptides by support vector machines,” PLoS Comput. Biol. 7(7) e1002101, which is incorporated herein by reference. For example, the cell-penetrating peptide can be derived from a phage display library. See, e.g., Shi et al. (2014) “A survey on ‘Trojan Horse’ peptides: Opportunities, issues and controlled entry to ‘Troy’,” J. Controlled Release, 194:53-70; Jarver et al. (2012) “Peptide-mediated cell and in vivo delivery of antisense oligonucleotides and siRNA,” Molecular Therapy-Nucleic Acids 1, e27; doi:10.1038/mtna.2012.18, which are incorporated herein by reference.

In some embodiments, the composition including bone-targeting complex 2000 further comprises a linker coupling the at least a portion of the nitric oxide synthase 2010 to at least one of the bone-targeting agent 2020 and the cell-penetrating means 2030. For example, the at least a portion of the nitric oxide synthase can be coupled or conjugated to the bone-targeting agent and/or the cell-penetrating means. In an aspect, the at least a portion of the nitric oxide synthase is coupled to the bone-targeting agent and/or the cell-penetrating peptide through a standard crosslinking reagent. In an aspect, the at least a portion of a nitric oxide synthase is coupled to the bone-targeting agent through a first crosslinking agent and to the cell-penetrating means through a second crosslinking agent. In an aspect, the bone-targeting agent is coupled to the at least a portion of a nitric oxide synthase through a first crosslinking agent and to the cell-penetrating means through a second crosslinking agent. In an aspect, to the cell-penetrating means is coupled to the at least a portion of a nitric oxide synthase through a first crosslinking agent and to the bone-targeting agent through a second crosslinking agent.

In an aspect, bone-targeting complex 2000 includes a linker that is a peptidyl linker, an oligomer, a ligand/receptor pair, an oligosaccharide, or an acyl chain. In an aspect, bone-targeting complex 2000 includes a linker that is a disulfide linker, a carbamate linker, an amide linker, an ester linker, or an ether linker. In an aspect, bone-targeting complex 2000 includes a linker that is a chemical crosslinker.

In an aspect, the linker comprises a cleavable linker. For example, bone-targeting complex 2000 can include a cleavable linker that is cleaved at some point after administration of the composition to a subject to release the at least a portion of a nitric oxide synthase from the bone-targeting agent and/or the cell-penetrating means. In an aspect, the cleavable linker is cleavable under extracellular conditions, releasing the at least a portion of a nitric oxide synthase from the bone-targeting agent and/or the cell-penetrating means in an extracellular environment. In an aspect, the cleavable linker is cleavable under intracellular conditions, releasing the at least a portion of a nitric oxide synthase from the bone-targeting agent and/or the cell-penetrating means in an intracellular environment (e.g., an intracellular enzymatic activity or in intracellular pH change). In an aspect, a first cleavable linker is cleaved in the extracellular environment while a second cleavable linker is cleaved in the intracellular environment.

In an aspect, bone-targeting complex 2000 includes a linker that is a stimulus-responsive cleavable linker. In an aspect, bone-targeting complex 2000 includes a linker that is at least one of an energy-responsive cleavable linker, a chemically-responsive cleavable linker, or an enzymatically-responsive cleavable linker.

In an aspect, bone-targeting complex 2000 includes an energy-responsive cleavable linker. Non-limiting examples of energy stimuli include electromagnetic energy, acoustic energy, magnetic energy, light energy, radiofrequency energy, and/or microwave energy. In an aspect, bone-targeting complex 2000 includes a linker that is at least one of a light-responsive cleavable linker, an ultrasound-responsive cleavable linker, or a heat-responsive cleavable linker. Non-limiting examples of light-responsive, ultrasound-responsive, and heat-responsive cleavable linkers have been described above herein.

In an aspect, bone-targeting complex 2000 includes a linker that is a chemically-responsive cleavable linker. For example, the stimulus-responsive cleavable linker can be responsive to a chemical reaction or condition (e.g., oxidizing conditions, reducing conditions, and/or pH conditions). In an aspect, bone-targeting complex 2000 includes a linker that is a pH-responsive cleavable linker. For example, the linker can include an acid-labile linker responsive to an acidic pH (e.g., an amino-sulfhydryl, thioether, hydrazone, semicarbazone, thiosemicarbazone, cis-aconitic amide, orthoester, acetal, ketal, or the like). In an aspect, the chemically-responsive cleavable linker is responsive to oxidation. For example, the chemically-responsive cleavable linker can be cleavable in response to the presence of superoxide. In an aspect, the chemically-responsive cleavable linker can include a disulfide group and be responsive to reducing conditions. Non-limiting examples of chemically-responsive cleavable linkers have been described above herein.

In an aspect, bone-targeting complex 2000 includes a linker that is an enzymatically-responsive cleavable linker. For example, the cleavable linker can be responsive to an enzymatic activity endogenous to the subject to whom the composition has been administered. For example, the cleavable linker can be responsive to an enzymatic activity specific to a target cell, e.g., a bone cell, to which the composition is directed or accumulates. For example, the enzymatically-responsive cleavable linker can include a peptidase- or protease-sensitive dipeptide or oligopeptide sensitive to cleavage by a peptidase or protease enzyme. In an aspect, the enzymatic stimulus, e.g., a peptidase or protease enzyme, is present in the intracellular environment, e.g., within a lysosome, endosome, or caveolea. In an aspect, the enzymatically-responsive cleavable linker is designed such that there is little or no cleavage of the linker in the plasma. In an aspect, the enzymatically-responsive cleavable linker is cleavable in response to a bone-specific peptidase or protease. For example, the enzymatically-responsive cleavable linker can include a peptide sequence cleavable by cathepsin K, a cysteine protease. For example, the enzymatically-responsive cleavable linker can include a peptide sequence cleavable by one or more matrix metalloproteinases. Non-limiting aspects of enzymatically-responsive cleavable linkers have been described above herein.

In an embodiment, the composition including bone-targeting complex 2000 further includes at least one carrier or excipient mixed with the bone-targeting complex to form at least one of a topical dosage form, an enteral dosage form, or a parenteral dosage form for delivery to a subject. Non-limiting aspects of carriers, excipients, and formulations have been described above herein.

FIG. 21 illustrates aspects of a method of treating a bone disorder. Method 2100 includes administering a bone-targeting complex to a subject in need of treatment for a bone disorder, the bone-targeting complex including at least a portion of a nitric oxide synthase, a bone-targeting agent, and a linker coupling the at least a portion of the nitric oxide synthase to the bone-targeting agent. In an aspect, method 2100 includes administering the bone-targeting complex to a human subject. In an aspect, method 2100 includes administering the bone-targeting complex to a mammalian subject.

In an aspect, method 2100 includes topically, enterally, or parenterally administering the bone-targeting complex to the subject in need of treatment for the bone disorder. For example, the method can include topically applying a composition including the bone-targeting complex to a skin surface in proximity to a bone and/or joint in need of treatment for a bone disorder, e.g., osteoarthritis or osteoporosis. For example, the method can include orally administering a liquid, tablet, or capsule including a composition including the bone-targeting complex to a subject in need of treatment for a bone disorder. For example, the method can include injecting a liquid composition including the bone-targeting complex into a subject in need of treatment for a bone disorder. In an embodiment, method 2100 includes injecting a liquid composition including the bone-targeting complex directly into a bone region in need of treatment.

In an aspect, method 2100 of treating a bone disorder further includes administering a bone-targeting complex to a subject in need of treatment for a bone disorder, the bone-targeting complex including at least a portion of a nitric oxide synthase, a bone-targeting agent, and a cleavable linker coupling the at least a portion of the nitric oxide synthase to the bone-targeting agent. In an aspect, the cleavable linker includes at least one of an energy-responsive cleavable linker, a chemically-responsive cleavable linker, or an enzymatically-responsive cleavable linker.

FIG. 22 illustrates aspects of a method of treating a bone disorder. Method 2200 includes administering a bone-targeting complex to a subject in need of treatment for a bone disorder, the bone-targeting complex including at least a portion of a nitric oxide synthase, a bone-targeting agent, and a cell-penetrating means. In an aspect, the cell penetrating means includes a lipid vesicle composition. In an aspect, the cell-penetrating means includes a cell-penetrating peptide.

In an aspect, method 2200 includes administering the bone-targeting complex to a human subject. In an aspect, method 2200 includes administering the bone-targeting complex to a mammalian subject.

In an aspect, method 2200 includes topically, enterally, or parenterally administering the bone-targeting complex to the subject in need of treatment for the bone disorder. For example, the method can include topically applying a composition including the bone-targeting complex to a skin surface in proximity to a bone and/or joint in need of treatment for a bone disorder, e.g., osteoarthritis or osteoporosis. For example, the method can include orally administering a liquid, tablet, or capsule including a composition including the bone-targeting complex to a subject in need of treatment for a bone disorder. For example, the method can include injecting a liquid composition including the bone-targeting complex into a subject in need of treatment for a bone disorder. In an embodiment, method 2200 includes injecting a liquid composition including the bone-targeting complex directly into a bone region in need of treatment.

In an aspect, method 2200 of treating a bone disorder further includes administering a bone-targeting complex to a subject in need of treatment for a bone disorder, the bone-targeting complex including at least a portion of a nitric oxide synthase, a bone-targeting agent, a cleavable linker coupling the at least a portion of the nitric oxide synthase to the bone-targeting agent, and a cell-penetrating means. In an aspect, the cleavable linker includes at least one of an energy-responsive cleavable linker, a chemically-responsive cleavable linker, or an enzymatically-responsive cleavable linker.

Non-limiting embodiments of the devices and methods described herein are presented in the following prophetic examples.

### Prophetic Example 1: A Bone Targeting Complex that Contains: an Inhibitor of Nitric Oxide Synthase Uncoupling, Midostaurin; a Cleavable Linker; and a Bone Targeting Agent, Hydroxyapatite Binding Protein

Oxidative stress and uncoupling of nitric oxide synthase (NOS) are associated with osteoporosis and bone resorption, and restoration of nitric oxide coupling and nitric oxide production in bone may inhibit bone resorption (see e.g., Wimalawansa, Ann. N.Y. Acad. Sci. 1192: 394-406, 2010 which is incorporated herein by reference). Uncoupling of nitric oxide synthase occurs when the enzyme fails to catalyze the production of nitric oxide from its substrate L-arginine, and instead, transfers an electron to oxygen (O2), generating superoxide (O2-). See e.g., Roe et al., Vascular Pharmacology 57: 168-172, 2012 which is incorporated herein by reference. Nitric oxide synthase uncoupling results in increased oxidative stress and reduced rates of nitric oxide production. In addition, superoxide may oxidize and inactivate tetrahydrobiopterin, an essential cofactor for nitric oxide synthase, thus exacerbating the uncoupling of synthase and reducing nitric oxide production.

A bone targeting complex to restore nitric oxide synthase coupling and nitric oxide production is constructed by coupling a bone targeting agent, hydroxyapaptite-binding peptide, to an inhibitor of nitric oxide synthase uncoupling, midostaurin, with a cleavable linker. The bone targeting complex binds to bone by virtue of hydroxyapatite-binding peptides which are selected using phage display techniques and made using in vitro automated synthesis. See e.g., U.S. Pat. No. 8,022,040 issued to Bertozzi et al. on Sep. 20, 2011 which is incorporated herein by reference. Hydroxyapatite-binding peptides approximately 7-12 amino acids in length which bind to crystalline hydroxyapatite are coupled with a cleavable linker that reacts with the amino terminal group on the peptide via N-hydroxysuccinimide. A heterobifunctional linker that is cleavable by thiol reagents (e.g., cysteine), amine-reactive and photo-reactive is available from Thermo Fisher Scientific, Waltham, Mass. (see e.g., NHS-SS-Diazirine, product no. 26175 in Thermo Scientific Pierce Crosslinking Technical Handbook, Ibid.) Next the covalent hydroxyapatite-binding peptide-linker product is coupled with midostaurin, an inhibitor of nitric oxide synthase uncoupling. Midostaurin is a protein kinase inhibitor which reduces reactive oxygen species (ROS) production and reverses NOS uncoupling (see e.g., Li et al., J. Am. Coll. Cardiol. 47: 2536-2544, 2006 and Forstermann and Li, Br. J. Pharmac. 164: 213-223, 2011 which are incorporated herein by reference). Photoactivation of diazirine with long wave UV light (330-370 nm) results in a reactive intermediate which forms a covalent bond with midostaurin. The resulting bone targeting complex contains in tandem: hydroxyapatite binding peptide-cleavable linker-midostaurin. The bone targeting complex can be purified using liquid chromatography and characterized by mass spectrometry.

The bone-targeting complex can be tested in a cell-based assay to assess whether the bone-targeting complex increases nitric oxide synthase enzymatic activity, i.e. increases nitric oxide production. Human mesenchymal stem cells (Cat. No. FC-0020, from Lifeline Cell Technology, Frederick, Md.) that have been differentiated to osteoblasts using an osteogenic growth medium (Cat. No. LM-0023, from Lifeline Cell Technology, Frederick, Md.) are used for the cell-based assay. A commercially available fluorometric cell-based nitric oxide detection assay using fluorescein amine methyl ester (Item #10009410, Cayman Chemical Co., Ann Arbor, Mich.) is used to measure nitric oxide product with and without the addition of the hydroxyapatite-binding peptide: Midostaurin complex.

The ability of the bone targeting complex to bind bone and to release midostaurin when a reducing agent (e.g., cysteine) is added may be tested in vitro and in vivo in animal models (see e.g., U.S. Pat. No. 6,133,320 Ibid. and U.S. Pat. No. 6,214,812 Ibid.).

### Prophetic Example 2: Construction of Bone-Targeting Complex with Tetrahydrobiopterin Linked to a Bisphosphonate Bone Targeting Agent

Tetrahydrobiopterin is an essential cofactor of nitric oxide synthase. Under oxidizing conditions, e.g., in the presence of superoxides, tetrahydrobiopterin is inactivated leading to decreased nitric oxide synthesis. To restore nitric oxide production, a bone-targeting complex is constructed by coupling a bone-targeting agent, bisphosphonate; a linker; and an activator of nitric oxide synthase, tetrahydrobiopterin. A bisphosphonate compound, alendronate, is the bone targeting agent. Alendronate binds strongly to solid calcium phosphate on the surface of bone, and covalently joins to a chemical linker via its primary amino group (see e.g., U.S. Pat. No. 7,288,535 issued to Garrett on Oct. 30, 2007 which is incorporated herein by reference). A heterobifunctional crosslinker is used link alendronate to tetrahydrobiopterin. For example a heterobifunctional crosslinker containing an amine-reactive succinimidyl ester (e.g., N-hydroxy succinimide (NHS)) at one end, and a sulfhydryl-reactive group (e.g., maleimide) at the other end. A heterobifunctional linker that reacts with amine and sulfhydryl groups is: N-(α-maleimidoacetoxy)-succinimide ester which is available from Thermo Fisher Scientific, Waltham, Mass. (see e.g., Thermo Scientific Pierce Crosslinking Technical Handbook,©2009 available online at: https://www.funakoshi.co.jp%2Fdownload%2Fcatalog%2FPCC4404.pdf&usg=AFQjCNFJ-s12tCCQtSwHaJ-NPpDN3pnOWQ&cad=rja which is incorporated herein by reference). Tetrahydrobiopterin is modified by addition of L-methionine to provide a sulfhydryl reactive with the crosslinker. Methods and reagents to derive amino acid derivatives of tetrahydrobiopterin are described (see e.g., U.S. Pat. No. 8,324,210 issued to Kakkis on Dec. 4, 2012 which is incorporated herein by reference). Sequential reactions of the crosslinker 1) with alendronate and 2) with tetrahydrobiopterin-L-methionine yields a bone targeting complex with the following structure: Alendronate-linker-tetrahydrobiopterin.

Methods to synthesize, purify and characterize the linked bone targeting complex are described (see e.g., Thermo Scientific Pierce Crosslinking Technical Handbook, Ibid.). For example, purification can be done using high pressure liquid chromatography (HPLC) and characterization can be done using mass spectrometry (see U.S. Pat. No. 8,324,210 Ibid.)

The purified bone-targeting complex can be tested in a cell-based assay to assess whether the complex activates nitric oxide synthase, i.e. increases nitric oxide production. Human mesenchymal stem cells (Cat. No. FC-0020, from Lifeline Cell Technology, Frederick, Md.) that have been differentiated to osteoblasts using an osteogenic growth medium (Cat. No. LM-0023, from Lifeline Cell Technology, Frederick, Md.) are used for the cell-based assay. A commercially available fluorometric cell-based nitric oxide detection assay using fluorescein amine methyl ester (Item #10009410, Cayman Chemical Co., Ann Arbor, Mich.) is used to measure nitric oxide product with and without the addition of the Tetrahydrobiopterin-Alendronate complex.

The tetrahydrobiopterin-alendronate complex is formulated for oral dosing as a tablet. For example, the complex can be combined with lactose, starch, talc, and magnesium stearate and pressed to form tablets.

### Prophetic Example 3: Method for Treating Osteoporosis with a Bone Targeting Complex

A postmenopausal subject with reduced bone density is treated with a bone targeting complex to inhibit further bone loss and to promote restoration of bone. The bone targeting complex, containing alendronate-linker-tetrahydrobiopterin is administered intravenously to target bone and deliver tetrahydrobiopterin to activate nitric oxide synthase and increase nitric oxide production.

The purified bone targeting complex, Alendronate-linker-tetrahydrobiopterin, is formulated to be suitable for intravenous (IV) injection and dosage and schedule of administration are determined. For example, a sterile, pyrogen-free, aqueous solution suitable for IV injection with respect to: pH, tonicity and stability. Suitable compositions may include: water, ethanol, glycerol, polyethylene glycol, and cellulose derivatives, and sterility may be obtained by sterile filtration and maintained by inclusion of a preservative (see e.g., U.S. Pat. No. 8,324,210 Ibid.). Pharmacokinetics and toxicology experiments in animals and man are used to determine the preferred dose and schedule for administration of the bone targeting complex. See, e.g., Remington's Pharmaceutical Sciences, 1435-712 (18th ed., Mack Publishing Co, Easton, Pa., 1990). For example, studies of tetrahydrobiopterin derivatives establish that intravenous administration of approximately 2 mg/kg of a tetrahydrobiopterin derivative to cynomologous monkeys is eliminated with kinetics similar to tetrahydrobiopterin. Preclinical studies in animals and clinical studies in man can determine safe and effective dosing and scheduling as well as the optimal route of administration. For example, radiolabeled (^{99m}Technetium conjugates and a gamma camera may be used to study biodistribution of the bone targeting complex (see e.g., U.S. Pat. No. 6,214,812 issued to Karpeisky et al. on Apr. 10, 2001 which is incorporated herein by reference). Methods to determine bone mineral density are known. For example, dual energy x-ray absorptiometric bone scan of vertebrae is used to determine the percent change in bone mineral density in grams/cm² (see e.g., U.S. Pat. No. 6,133,320 issued to Yallampalli et al. on Oct. 17, 2000 which is incorporated herein by reference).

### Prophetic Example 4: A Bone Targeting Complex to Deliver Nitric Oxide Synthase

To provide nitric oxide to bone a molecular complex is constructed that targets delivery of nitric oxide synthase to bone and promotes production of nitric oxide in situ. A molecular complex to deliver nitric oxide synthase contains a bone-targeting agent, a linker, nitric oxide synthase, and a cell-penetrating peptide. The bone targeting complex binds to bone by virtue of hydroxyapatite-binding peptides which are selected using phage display techniques and made using in vitro automated synthesis. See e.g., U.S. Pat. No. 8,022,040 issued to Bertozzi et al. on Sep. 20, 2011 which is incorporated herein by reference. The cell-penetrating peptide facilitates entry of the bone-targeting complex through the membrane and into a cell.

Nitric oxide synthase is produced using recombinant DNA methods and purified prior to conjugation with other components of the bone-targeting complex. In this example, human inducible nitric oxide synthase is produced in mammalian cells, e.g., HepG2 cells, using an adenovirus vector that expresses inducible nitric oxide synthase under the control of the human cytomegalovirus promoter (see e.g., Fishbein et al., Proc. Natl. Acad. Sci. USA, 103: 159-164, 2006 which is incorporated herein by reference). The recombinant nitric oxide synthase is purified from cell homogenates using affinity chromatography (see e.g., Nakane et al, Bioch. Bioph. Res. Comm., 206: 511-517, 1995 which is incorporated herein by reference) and conjugated to the bone targeting complex. Multiple cysteines present in nitric oxide synthase are not essential for enzyme activity (see e.g., Stuehr, Bioch. Bioph. Acta, 1411: 217-230, 1999 which is incorporated herein by reference) and may be used for coupling with a sulfhydryl-reactive moiety of a crosslinker.

Hydroxyapatite-binding peptides approximately 7-12 amino acids in length is generated as described in Example 1 and coupled with a cleavable linker that reacts with the amino terminus of the peptide via N-hydroxysuccinimide. An exemplary heterobifunctional linker Sulfo-SMCC (Sulfosuccinimidyl 4-(N-maleimidomethyl)cyclohexane-1-carboxylate) that includes an amine-reactive moiety and a sulfhydryl-reactive moiety is available from Thermo Fisher Scientific, Waltham, Mass. (see e.g., NHS-SS-Diazirine, product no. 26175 in Thermo Scientific Pierce Crosslinking Technical Handbook, Ibid.). The hydroxyapatite-binding peptide is reacted with

Sulfo-SMCC per the manufacturer's instructions. In general, the less stable amine-reactive moiety of Sulfo-SMCC will preferentially covalently interact with amino groups on the hydroxyapatite-binding peptide.

A cell-penetrating peptide, e.g., TAT (HIV-1) (48-61), is obtained from a commercial source (Cat. #068-26 from Phoenix Pharmaceuticals, Inc., Burlingame, Calif.). The TAT peptide is reacted with Sulfo-SMCC per the manufacturer's instructions. In general, the less stable amine-reactive moiety of Sulfo-SMCC will preferentially covalently interact with amino groups on the TAT peptide.

Sulfo-SMCC modified hydroxyapatite-binding peptide and Sulfo-SMCC modified TAT peptide are subsequently incubated with the purified nitric oxide synthase. The sulfhydryl-reactive moiety of the Sulfo-SMCC linker reacts with cysteine residues present in the purified nitric oxide synthase. The resulting bone-targeting complex is purified using liquid chromatography and characterized by mass spectrometry.

The bone-targeting complex can be tested in vitro for nitric oxide synthase enzymatic activity, i.e. nitric oxide production. For example, nitric oxide synthase activity is determined by quantifying the conversion of L-arginine into L-citrulline. Briefly, 2 micrograms of the bone-targeting complex including nitric oxide synthase linked to bisphosphonate is incubated for 5-10 minutes at 37 degrees centigrade in HEPES buffer (pH 7.4) containing DTPA (0.1 mmol/1), CaCl₂ (0.2 mmol/1), calmodulin (20 micrograms/ml), NADPH (0.5 mmol/1), FMN (1 micromoles/l), FAD (1 micromole/l), glutathione (100 micromole/l), bovine serum albumin (200 micrograms/ml), L-arginine (100 micromole/l), and L-tritiated-arginine (3.7 KBq). All measurements were performed in triplicate. After correction for nonspecific activity, nitric oxide synthase activity is calculated from the percent conversion of tritiated-arginine into tritiated-citrulline and expressed as nmoles per mg protein per min.

The bone-targeting complex can be tested in a cell-based assay for increased nitric oxide synthase enzymatic activity, i.e. increased nitric oxide production, as a result of incubating cells with the bone-targeting complex. Human mesenchymal stem cells (Cat. No. FC-0020, from Lifeline Cell Technology, Frederick, Md.) that have been differentiated to osteoblasts using an osteogenic growth medium (Cat. No. LM-0023, from Lifeline Cell Technology, Frederick, Md.) are used for the cell-based assay. A commercially available fluorometric cell-based nitric oxide detection assay using fluorescein amine methyl ester (Item #10009410, Cayman Chemical Co., Ann Arbor, Mich.) is used to measure nitric oxide product with and without the addition of the hydroxyapatite-binding peptide:nitric oxide synthase:TAT complex.

The hydroxyapatite-binding peptide:nitric oxide synthase:TAT complex is formulated for injection proximal to or into bone. For example, the formulation can include a sterile aqueous solution including a phosphate buffer, polysorbate 80, and sucrose.

One skilled in the art will recognize that the herein described component, devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components, devices, and objects should not be taken as limiting.

With respect to the use of substantially any plural and/or singular terms herein, the plural can be translated to the singular and/or from the singular to the plural as is appropriate to the context and/or application. The various singular/plural permutations are not expressly set forth herein for sake of clarity.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

While particular aspects of the present subject matter described herein have been shown and described, changes and modifications can be made without departing from the subject matter described herein and its broader aspects and, therefore, the appended claims are to encompass within their scope all such changes and modifications as are within the true spirit and scope of the subject matter described herein. Terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition, comprising:
a bone-targeting complex including
at least a portion of a nitric oxide synthase;
a bone-targeting agent; and
a linker coupling the at least a portion of the nitric oxide synthase to the bone-targeting agent.

2. (canceled)

3. The composition of claim 1, wherein the at least a portion of the nitric oxide synthase is conjugated to a first end of the linker and the bone-targeting agent is conjugated to a second end of the linker.

4. The composition of claim 1, wherein the at least a portion of the nitric oxide synthase comprises at least a portion of recombinant nitric oxide synthase.

5. The composition of claim 1, wherein the at least a portion of the nitric oxide synthase comprises at least a portion of endothelial nitric oxide synthase.

6. The composition of claim 1, wherein the at least a portion of the nitric oxide synthase comprises at least a portion of neuronal nitric oxide synthase.

7. The composition of claim 1, wherein the at least a portion of the nitric oxide synthase comprises at least a portion of inducible nitric oxide synthase.

8. The composition of claim 1, wherein the at least a portion of the nitric oxide synthase comprises a homodimer of the at least a portion of the nitric oxide synthase.

9. The composition of claim 1, wherein the bone-targeting agent comprises bisphosphonate.

10.-12. (canceled)

13. The composition of claim 1, wherein the bone-targeting agent comprises a hydroxyapatite-binding polypeptide.

14. The composition of claim 1, wherein the bone-targeting agent comprises a bone morphogenetic protein.

15. The composition of claim 1, wherein the linker comprises a cleavable linker.

16. The composition of claim 15, wherein the cleavable linker comprises a stimulus-responsive cleavable linker.

17. The composition of claim 16, wherein the stimulus-responsive cleavable linker comprises an energy-responsive cleavable linker.

18. (canceled)

19. The composition of claim 16, wherein the stimulus-responsive cleavable linker comprises a chemically-responsive cleavable linker.

20. The composition of claim 19, wherein the chemically-responsive cleavable linker comprises a pH-responsive cleavable linker.

21. The composition of claim 16, wherein the stimulus-responsive cleavable linker comprises an enzymatically-responsive cleavable linker.

22. The composition of claim 1, further comprising a cell-penetrating means associated with the bone-targeting complex.

23. The composition of claim 22, wherein the cell-penetrating means comprises a lipid vesicle formulation.

24. The composition of claim 22, wherein the cell-penetrating means comprises a cell-penetrating peptide.

25. The composition of claim 24, wherein the cell-penetrating peptide comprises an arginine-rich peptide, a lysine-rich peptide, or a combined arginine-lysine-rich peptide.

26. The composition of claim 24, wherein the cell-penetrating peptide comprises a hydrophilic cell penetrating peptide.

27. The composition of claim 24, wherein the cell-penetrating peptide comprises an amphiphilic cell penetrating peptide.

28. The composition of claim 24, wherein the cell-penetrating peptide comprises a HIV-1 Tat peptide, a penetratin peptide, a transportan peptide, or derivatives thereof.

29. The composition of claim 1, further comprising at least one carrier or excipient mixed with the bone-targeting complex to form at least one of a topical dosage form, an enteral dosage form, or a parenteral dosage form for delivery to a subject.

30. A composition, comprising:
a bone-targeting complex including
at least a portion of a nitric oxide synthase;
a bone-targeting agent; and
a cell-penetrating means.

31. The composition of claim 30, wherein the at least a portion of the nitric oxide synthase comprises at least a portion of endothelial nitric oxide synthase, neuronal nitric oxide synthase, or inducible nitric oxide synthase.

32. The composition of claim 30, wherein the bone-targeting agent comprises at least one of bisphosphonate, a hydroxyapatite-binding peptide, or a bone morphogenetic protein.

33. The composition of claim 30, wherein the cell-penetrating means comprises a lipid vesicle formulation.

34. The composition of claim 30, wherein the cell-penetrating means comprises a cell penetrating peptide.

35. The composition of claim 30, further comprising a linker coupling the at least a portion of the nitric oxide synthase to at least one of the bone-targeting agent and the cell-penetrating means.

36. (canceled)

37. The composition of claim 35, wherein the linker comprises a stimulus-responsive cleavable linker.

38. The composition of claim 37, wherein the stimulus-responsive cleavable linker comprises at least one of an energy-responsive cleavable linker, a chemically-responsive cleavable linker, or an enzymatically-responsive cleavable linker.

39. (canceled)

40. A method of treating a bone disorder, comprising:
administering a bone-targeting complex to a subject in need of treatment for a bone disorder, the bone-targeting complex including at least a portion of a nitric oxide synthase, a bone-targeting agent, and a cell-penetrating means.
